(19) United States
(12) Patent Application Publication
(10) Pub. No.: US 2026/0062414 A1
(43) Pub. Date: Mar. 5, 2026

(54) NOVEL COCRYSTALS OF METHYLXANTHINES, THEIR POLYMORPHS AND PROCESS THEREOF

(71) Applicant: FERTIS INDIA PVT LTD, Punjagutta, Hyderabad, Telangana (IN)

(72) Inventors: Rahul Raju KANUMURU, Punjagutta, Hyderabad, Telangana (IN); Shaji George KOCHUMALAYIL, Punjagutta, Hyderabad, Telangana (IN); Ravikumar SURANENI, Punjagutta, Hyderabad, Telangana (IN); Murali Mohanarao VANA, Punjagutta, Hyderabad, Telangana (IN)

(73) Assignee: FERTIS INDIA PVT LTD, Punjagutta, Hyderabad, Telangana (IN)

(21) Appl. No.: 19/107,437

(22) PCT Filed: Aug. 28, 2023
(86) PCT No.: PCT/IN2023/050810

§ 371 Date: Feb. 27, 2025

(30) Foreign Application Priority Data
Aug. 30, 2022 (IN) 202241049555

(51) Int. Cl. C07D 473/08; A61K 31/05; A61K 31/09; A61K 31/522; C07C 39/21; C07C 43/23; C07D 473/10; C07D 473/12; C07D 473/14

(52) U.S. Cl. CPC C07D 473/08; A61K 31/05; A61K 31/09; A61K 31/522; C07C 39/21; C07C 43/23; C07D 473/10; C07D 473/12; C07D 473/14; C07B 2200/13

(57) ABSTRACT

The present invention relates to cocrystals of Methylxanthines with Stilbenoids as coformers, their polymorphic forms and to the process for preparation thereof. The debittering of Methylxanthines is carried out by co-crystallization using Stilbenoids as coformers. The invention further relates to the preparation of polymorphic forms of debittered cocrystals and their use in nutraceutical and pharmaceutical compositions.

### TECHNICAL FIELD

The present invention relates to cocrystals of Methylxanthines with Stilbenoids as coformers, their polymorphic forms and to the process for preparation thereof.

The debittering of Methylxanthines is carried out by co-crystallization using Stilbenoids as coformers. The invention further relates to the preparation of polymorphic forms of debittered cocrystals and their use in nutraceutical and pharmaceutical compositions.

Background and prior art: Methylxanthines like Caffeine. Theacrine, Theobromine, Methylliberine, Theophylline, Paraxanthine are purine alkaloids purported to have similar neuro-energetic effects. These were identified in the seeds and leaves of various Coffea species. Initially, Caffeine accumulates in young leaves of coffee plants but that is gradually replaced by Theacrine, Theobromine and Methylliberine, when it became adult leaves.

Pterostilbene chemically known as trans-3,5-dimethoxy-4′-hydroxystilbene is a naturally occurring stilbene, being found in nature such as in number of tree barks and a variety of berries, including grapes, as well as plants commonly used in traditional folk medicine and structurally related to Resveratrol. It has been characterized as a nutraceutical with potential antioxidant, anticancer, anti-inflammatory, pro-apoptotic, antineoplastic, cytoprotective properties and further reported to decrease plasma glucose levels and also lowers the LDL/HDL cholesterol ratio. It is reported that upon administration, Pterostilbene exerts its antioxidant activity by scavenging reactive oxygen species (ROS), thereby preventing oxidative stress and ROS-induced cell damage.

The use of Pterostilbene to ameliorate oxidative stress and improve working memory and compositions containing Pterostilbene are described in US2009/0069444. Due to the perceived health benefits of Pterostilbene; there is an increase in the consumption of foods that contain this compound, such as berries and grapes.

WO2011097372A3 discloses cocrystals of Pterostilbene which includes Pterostilbene: Caffeine cocrystal, Pterostilbene: Carbamazepine cocrystal, Pterostilbene: Glutaric acid cocrystal, and Pterostilbene: Piperazine cocrystal.

U.S. Pat. No. 9,468,645B2 discloses stable aqueous compositions, for oral administration, of at least one biologically-active form of a purine alkaloid compound structurally related to Caffeine, which compositions comprise of a) 1,3,7,9-tetramethyl uric acid (Theacrine), (O(2), 2-Methoxy-1,9-dimethyl-7H-purine-6,8-dione (Liberine) and (O(2), 1,7,9-trimethyl uric acid (Methylliberine), collectively referred to as Theacrine species, wherein a basic uric acid structure has been partially or fully methylated in 2, 3 or 4 positions; and b) at least one aqueous buffered solution having a pH range of about 1.5-9.0.

EP3068240A1 discloses a dietary supplement that comprises Theacrine and optionally other active ingredients that modulate the effects of Theacrine.

US20210100266 discloses energy supplement compositions comprising Caffeine and Methylliberine.

Although Methylxanthines are known to have very good health benefits, however, the usage is limited due to their bitter taste and hence required to mask the bitterness or to make it consumable by debittering the bitterness of these nutraceuticals to improve palatability. Unacceptable palatability is a major problem in the development of a nutraceutical formulation, as it often impedes patient adherence. Masking the bitter taste of methylxanthines is, therefore, a major goal in the development of a nutraceutical formulation and is widely recognized as a key aspect in modem drug development.

The co-crystallization of two or more pure nutraceutical compounds by crystal engineering to create a new functional material with improved taste and bioavailability is of great industrial interest for both pharmaceutical and food industries. Therefore, there remains a need in the art to provide compositions comprising Methylxanthines, which taste good, are devoid of bitterness, and improved bioavailability.

### Objectives of the Invention

Therefore, it is an objective of the present invention to provide cocrystals of Methylxanthines with Stilbenoids, which tastes good and is devoid of bitterness.

It is another objective of the present invention to provide cocrystals of Methylxanthines, using Stilbenoids more specifically Pterostilbene and Resveratrol as a crystal former, for masking the taste of Methylxanthines.

It is a further objective of the invention to provide process for the preparation of cocrystals of Methylxanthines using Stilbenoids more specifically Pterostilbene and Resveratrol as a crystal former and compositions comprising the same.

It is a further objective of the present invention to provide process for the preparation of polymorphic forms of cocrystals of Methylxanthines with Pterostilbene and Resveratrol.

### SUMMARY OF THE INVENTION

In line with the above objectives, the present invention provides cocrystals of Methylxanthines with Stilbenoids as coformers, and to polymorphic forms thereof, wherein said cocrystal exhibits a pleasant taste and is free from the bitterness of methylxanthines.

The methylxanthines are selected from Theacrine, Theobromine, Methylliberine, Caffeine, Paraxanthine and Theophylline.

The Stilbenoids are selected from Hydroxy derivatives of Styrenes, preferably Pterostilbene and Resveratrol.

In a preferred aspect, the present invention provides the debittered cocrystals of Methylxanthines with Stilbenoids, comprising.
(a) Theacrine: Pterostilbene
(b) Theobromine: Pterostilbene
(c) Methylliberine: Pterostilbene
(d) Paraxanthine: Pterostilbene
(e) Caffeine: Pterostilbene and
(f) Theophylline: Resveratrol
wherein said cocrystal exhibits a pleasant taste and is free from the bitterness of Methylxanthines.

In another aspect, the Methylxanthines and the co-formers present in the cocrystal are in a molar ratio of 1:4 to 4:1 preferably 1:1.

In yet another aspect, the present invention provides novel cocrystals of Theacrine and Pterostilbene with cocrystal polymorphic forms Form 1, Form II, Form III, Form IV and Form V and to the processes for the preparation thereof.

Accordingly, the present invention provides crystalline Theacrine: Pterostilbene cocrystal polymorphic Form I, which is characterized by an X-ray powder diffraction pattern having one or more peaks with relative intensity at about 12.78 (100%), 14.18 (90.2%), 14.69 (17.9%), 15.55 (19.0%), 17.45 (11.8%), 19.04 (9.1%), 22.28 (12.0%), 23.46 (14.4%), 27.90 (5.0%), 28.80 (5.6%), 29.50 (7.3%), 31.50 (7.6%), 33.04 (10.1%), 38.90 (14.2%), 41.02 (6.1%), 41.93 (4.7%), 53.16 (4.5%). 72.73 (8.4%)±0.2° 2θ.

According to another aspect, the invention provides crystalline Theacrine: Pterostilbene cocrystal polymorphic Form II which is characterized by an X-ray powder diffraction pattern having one or more peaks with relative intensity at about 10.98 (4.8%), 13.06 (33.6%), 14.45 (65.4%), 15.09 (38.5%), 15.85 (32.3%), 17.71 (6.5%), 19.18 (13.9%), 20.21 (28.7%), 21.44 (6.8%), 22.58 (11.1%), 23.73 (9.5%), 26.40 (59.7%), 27.21 (100%), 28.29 (10.0%), 29.12 (2.7%), 29.87 (3.6%), 31.76 (5.8%), 32.10 (6.2%), 33.33 (6.1%), 35.73 (1%), 37.02 (1.5%), 39.20 (9.0%), 40.66 (1.0%), 45.00 (1.4%), 46.38 (1.8%), 47.41 (1.2%), 48.05 (1.7%), 54.61 (2.8%), 55.46 (2.4%) and 72.69 (2.5%)±0.2° 2θ.

In another aspect, the invention provides crystalline Theacrine: Pterostilbene cocrystal polymorphic Form III, which is characterized by an X-ray powder diffraction pattern having one or more peaks with relative intensity at about 12.90 (64.2%), 14.32 (100%), 15.64 (13.3%), 17.45 (8.2%), 19.17 (6.6%), 21.69 (10.5%), 22.38 (7.9%), 23.58 (10.0%), 25.07 (7.0%), 26.19 (6.1%), 26.98 (9.6%), 28.86 (1.9%), 29.59 (2.8%), 31.58 (2.7%), 33.12 (3.7%), 38.98 (7%), 41.10 (1.4%), 43.62 (1.1%), 47.80 (1.0%), and 72.70 (2.7%)±0.2° 2θ.

In yet another aspect, the present invention provides crystalline Theacrine: Pterostilbene cocrystal polymorphic Form IV, which is characterized by an X-ray powder diffraction pattern having one or more peaks with relative intensity at about 12.98 (100%), 14.38 (97.8%), 15.72 (18.6%), 17.66 (14.9%), 19.38 (10.3), 21.72 (7.5%), 22.47 (9.1%), 23.69 (12.8%), 25.14 (8.1%), 26.16 (11.6%), 27.00 (17.2%), 28.03 (2.5%), 29.69 (4.4%), 30.83 (1.1%), 31.66 (4.3%), 33.25 (7.2%), 36.88 (1.1%), 39.12 (11.6%), 41.21 (2.1%), 43.62 (1.3%), 44.06 (1.7%), 44.83 (1.3%), 45.99 (1.0%), 47.30 (1.1%), 47.95 (1.9%), 49.04 (1.0%), 53.36 (1.5%), 72.68 (3.3%) and 88.31 (1.0%)±0.2° 2θ.

In another aspect, the present invention provides crystalline Theacrine: Pterostilbene cocrystal polymorphic Form V, which is characterized by an X-ray powder diffraction pattern having one or more peaks with relative intensity at about 12.83 (17.0%), 14.33 (100%), 15.72 (18.8%), 19.15 (3.5%), 20.06 (3.5%), 22.10 (8.8%), 23.55 (6.5%), 27.17 (5.8%), 31.73 (4.8%), 33.71 (2.0%), 37.88 (1.4%), 39.19 (6.2%), 41.07 (1.4%), 41.54 (2.2%), 43.86 (1.0%)±0.2° 2θ.

In yet another aspect, the present invention provides crystalline Theobromine: Pterostilbene cocrystal polymorphic Form I, which is characterized by an X-ray powder diffraction pattern having one or more peaks with relative intensity at about 11.75 (30.21%), 13.38 (63.19%), 15.37 (7.89%), 17.31 (10.2%), 19.35 (95.4%), 23.46 (44.3%), 26.98 (100%), 29.35 (14.1%), 31.65 (6.5%), 34.30 (2.2%), 35.93 (6.2%), 39.15 (5.4%), 40.67 (3.6%), 42.08 (3.2%), 44.61 (2.9%), 48.89 (2.4%), 50.06 (2.9%), 52.53 (2.4%), 55.83 (2.2%), 72.73 (6.96%), 88.42 (2.4%)±0.2° 2θ.

In yet another aspect, the present invention provides Methylliberine and Pterostilbene cocrystals polymorphic forms Form 1, Form II, Form III, and Form IV and processes for the preparation thereof.

In another aspect, the present invention provides crystalline Methylliberine: Pterostilbene cocrystal polymorphic Form I, which is characterized by an X-ray powder diffraction pattern having one or more peaks at about 12.25 (87.7%), 13.33 (100%), 15.52 (24.8%), 16.44 (6.6%), 17.98 (6.0%), 19.99 (16.5%), 21.12 (8.6%), 21.99 (5.2%), 23.93 (4.5%), 25.44 (6.6%), 27.05 (11.1%), 28.76 (3.9%), 34.75 (3.8%), 37.81 (5.8%), 40.49 (3.0%), 42.78 (4.9%), 44.64 (3.9%), 72.73 (5.8%)±0.2° 2θ.

In another aspect, the present invention provides crystalline Methylliberine: Pterostilbene cocrystal polymorphic Form II, which is characterized by an X-ray powder diffraction pattern having one or more peaks with relative intensity at about 12.43 (68.3%), 13.49 (100%), 15.65 (11.9%), 16.60 (11%), 18.12 (4.6%), 20.13 (15.3%), 21.30 (7.2%), 22.16 (6.2%), 23.34 (1.5%), 24.08 (4.4%), 25.69 (17%), 26.91 (16.8%), 27.31 (22.4%), 29.91 (1.3%), 30.82 (1.2%), 31.29 (1.6%), 34.51 (1.3%), 36.24 (1.4%), 37.94 (3.7%), 42.82 (1.0%), 44.67 (1.1%), 72.70 (2.5%)±0.2° 2θ.

In an aspect, the present invention provides crystalline Methylliberine: Pterostilbene cocrystal polymorphic Form III, which is characterized by an X-ray powder diffraction pattern having one or more peaks with relative intensity at about 13.20 (100%), 15.35 (16.1%), 16.50 (11.8%), 17.90 (3.2%), 19.88 (13.3%), 20.83 (3.9%), 22.06 (5.0%), 23.05 (1.3%), 23.88 (4.1%), 25.60 (14.9%), 36.03 (1.1%), 42.05 (1.2%), and 72.67 (7.7%)±0.2° 2θ.

In another aspect, the present invention provides crystalline Methylliberine: Pterostilbene cocrystal polymorphic Form IV, which is characterized by an X-ray powder diffraction pattern having one or more peaks with relative intensity at about 12.00 (43.3%), 12.48 (91.5%), 13.63 (84.8%), 15.32 (11.0%), 16.77 (24.7%), 18.34 (10.8%), 19.02 (11.1%), 20.22 (20.4%), 21.44 (16.9%), 22.32 (14.8%), 23.34 (3.9%), 24.24 (10.5%), 25.84 (100%), 27.48 (81.2%), 33.31 (2.7%), 34.78 (5.1%), 35.01 (4.9%), 36.44 (3.5%), 38.10 (8.2%), 40.78 (1.5%), 43.12 (4.9%), 46.52 (2.7%), 47.75 (1.7%), 49.85 (1.4%)±0.2° 2θ.

In yet another aspect, the present invention provides Novel polymorphic forms of Caffeine and Pterostilbene cocrystals Form 1, Form II, Form III, and Form IV and processes for the preparation thereof.

Accordingly, the present invention provides crystalline Caffeine: Pterostilbene cocrystal polymorphic Form I, which is characterized by an X-ray powder diffraction pattern having one or more peaks with relative intensity at about 11.90 (28.6%), 15.03 (100%), 16.81 (51.2%), 19.06 (53.5%), 21.65 (44.4%), 25.81 (94.2%), 26.35 (91.04%), 28.16 (10.4%), 29.54 (6.7%), 31.32 (4.8%), 32.52 (10.3%), 35.74 (3.6%), 36.99 (3.8%), 39.36 (3.7%), 40.93 (7.3%), 43.66 (5.7%), 45.45 (3.6%), 49.83 (2.1%), 72.74 (8.5%) and 88.35 (3.3%)±0.2° 2θ.

In another aspect, the present invention provides crystalline Caffeine: Pterostilbene cocrystal polymorphic Form II, which is characterized by an X-ray powder diffraction pattern having one or more peaks with relative intensity at about 12.83 (2.19%), 17.04 (5.43%), 19.41 (5.24%), 22.25 (6.28%), 26.31 (100%), 28.67 (3.8%), 30.04 (1.6%), 32.96 (2.3%), 41.34 (1.1%), and 53.94 (1.7%)±0.2° 2θ.

In another aspect, the present invention provides crystalline Caffeine: Pterostilbene cocrystal polymorphic Form III, which is characterized by an X-ray powder diffraction pattern having one or more peaks with relative intensity at about 10.53 (18.3%), 12.18 (4.9%), 14.01 (23.04%), 15.56 (25.3%), 17.22 (21.0%), 19.35 (16.7%), 22.13 (13.7%), 24.02 (4.3%), 26.51 (100%), 28.57 (20.4%), 30.22 (4.0%), 31.82 (4.1%), 36.23 (1.1%), 43.3 (1.8%), 46.52 (1.1%), 53.52 (1.1%) and 72.72 (2.2%)±0.2° 2θ.

In another aspect, the present invention provides crystalline Caffeine: Pterostilbene cocrystal polymorphic Form IV, which is characterized by an X-ray powder diffraction pattern having one or more peaks with relative intensity at about 10.49 (100%), 12.24 (18.5%), 13.92 (35.3%), 14.18 (53.8%), 15.49 (41.5%), 15.60 (63.8%), 17.08 (36.3%), 19.3 (24.9%), 19.5 (60.2%), 22.13 (35.6%), 23.77 (12.7%), 26.06 (79.5%), 26.7 (94.8%), 28.51 (17.7%), 30.00 (13.0%), 32.48 (10.0%), 36.36 (10.3%), 38.15 (12.0%), 40.00 (8.1%), 41.28 (10.4%), 43.26 (7.5%), 46.34 (14.8%), 50.19 (8.5%), 53.95 (9.2%), 56.00 (5.7%)±0.2° 2θ.

In yet another aspect, the present invention provides novel cocrystals of Paraxanthine with Pterostilbene and process for preparation thereof.

Accordingly, the present invention provides crystalline Paraxanthine: Pterostilbene cocrystal characterized by an X-ray powder diffraction pattern having one or more peaks with relative intensity at about 11.31 (12.8%), 15.88 (71.8%), 19.86 (18.7%), 21.28 (26.0%), 22.52 (33.6%), 24.9 (83.2%), 26.96 (100%), 29.00 (12.3%), 29.21 (19.3%), 30.41 (14.3%), 32.45 (9.9%), 38.8 (6.5%), 40.68 (5.2%), 44.84 (4.9%), 50.55 (3.6%), 55.48 (4.4%). 0.2° 20.

In yet another aspect, the present invention provides novel cocrystals of Theophylline: Resveratrol and process for preparation thereof.

Accordingly, the cocrystals of Theophylline: Resveratrol characterized by an X-ray powder diffraction pattern having one or more peaks with relative intensity at about: 10.30 (9.4%), 12.40 (30.7%), 13.81 (100%), 15.21 (9.4%), 16.27 (14.1%), 17.21 (4.5%), 19.22 (21.1%), 20.76 (13.0%), 22.30 (8.4%), 23.52 (8.4%), 24.84 (47.9%), 25.36 (33.6%), 26.64 (7.4%), 28.26 (8.6%), 30.68 (8.9%), 32.88 (3.3%), 34.96 (1.1%), 36.46 (1.5%), 37.61 (1.1%), 38.45 (2.0%), 39.84 (1.4%), 42.16 (2.3%), 45.91 (1.3%), 52.26 (1.3%), 72.73 (4.1%) and 88.35 (1.2%)±0.2° 2θ.

In an aspect, the present invention provides a process for the preparation of said debittered cocrystals of Methylxanthines and their polymorphic forms comprising of;
(a) Dissolving the coformer in a solvent at 35-55° C. to obtain clear solution followed by addition of Methylxanthines.
(b) Stirring the above mixture at a temperature of 40-60° C. to obtain a clear solution;
(c) Gradually cooling the above solution to ambient temperature and maintaining said temperature for a period of 10-20 h to allow the co-crystallization; and
(d) Separating the co-crystal thus formed on standing followed by filtering, washing and drying.

The solvent for the process is selected from water, C1-C5 alcohols, lower organic acids such as formic acid, acetic acid, propanoic acid, butyric acid, ethers, esters, ketones alone or mixtures thereof.

In an alternate aspect, the method of preparation of debittered cocrystals comprises mechanical grinding of a mixture of Methylxanthines and Stilbenoids in a molar ratio of 1:4 to 4:1 preferably 1; 1 to obtain the desired cocrystal.

In another aspect, the present invention provides a process for masking the bitterness of Methylxanthines comprising;
(a) Dissolving the coformer in a solvent at 35-55° C. to obtain clear solution followed by addition of Methylxanthines.
(b) Stirring the above mixture at a temperature of 40-60° C. to obtain a clear solution;
(c) Gradually cooling the above solution to ambient temperature and maintaining said temperature for a period of 10-20 h to allowing the co-crystallization; and
(d) Separating the co-crystal thus formed on standing followed by filtering, washing and drying.

In yet another aspect, the method of masking the bitterness of Methylxanthines selected from Theacrine, Theobromine, Methylliberine, Caffeine, Paraxanthine or Theophylline comprises mechanical grinding of a mixture of methylxanthines and Pterostilbene or Resveratrol in a molar ratio of 1:4 to 4:1, preferably 1:1 to obtain the desired debittered cocrystal.

In an aspect, the mechanical grinding is carried out in the presence of a solvent or in the absence of a solvent. In yet another aspect, coformer slurring is carried out in an alcoholic solvent or mixture of alcoholic solvents and water or in organic acids.

In yet another aspect, the invention provides nutraceutical/pharmaceutical compositions comprising co-crystals of Theacrine, Theobromine, Methylliberine, Caffeine, Paraxanthine and Theophylline and their polymorphic forms in association with one or more suitable carriers, wherein, the coformer selected from Pterostilbene or Resveratrol masks the bitterness of Methylxanthines by blocking the sensory receptors responsible for taste.

### DESCRIPTION OF THE FIGURES

FIG. 1. Depicts Dissolution data of different Theacrine-Pterostilbene cocrystal systems plotted in accordance to 0^{th} order equation (1.A.), 1st order equation (1.B.), and Higuchi square root equation (1.C.)

FIG. 2. Dissolution data of different Methylliberine-Pterostilbene cocrystal systems plotted in accordance to 0^{th} order equation (2.A.), 1^{st} order equation (2.B.), and Higuchi square root equation (2.C.)

FIG. 3. Dissolution data of different Caffeine-Pterostilbene cocrystal systems plotted in accordance to 0^{th} order equation (3.A.), 1^{st} order equation (3.B.), and Higuchi square root equation (3.C.)

FIG. 4. Dissolution data of Theophylline-Resveratrol cocrystal system plotted in accordance to 0^{th} order equation (4.A.), 1^{st} order equation (4.B.), and Higuchi square root equation (4.C.)

FIG. 5. Dissolution data of Theobromine-Pterostilbene cocrystal system plotted in accordance to 0^{th} order equation.

FIG. 6. % availability of active ingredient as a function of time for various cocrystal systems

### DETAILED DESCRIPTION OF THE INVENTION

The foregoing descriptions of specific embodiments of the present invention have been presented for the purposes of illustration and description. They are not intended to be exhaustive or to limit the invention to the precise forms disclosed, and obviously many modifications and variations are possible in light of the above teaching.

Pterostilbene, Resveratrol, Theacrine, Theobromine, Methylliberine, Paraxanthine Caffeine and Theophylline used in the present invention are commercially available synthetic compounds.

The terms coformer, cocrystal, nutraceutical and pharmaceutical used in the application have the same meaning as used in scientific literature.

Taste assessment is an increasingly important aspect of development of any oral formulations. The bitterness is the most problematic due to its intuitive association with toxicity. There are various taste-masking techniques which may be used to inhibit bitter taste such as polymer coating, cocrystal formation or complexation with cyclodextrins etc.

The present inventors have surprisingly found that when the Methylxanthines such as Theacrine, Theobromine, Methylliberine, Caffeine and Paraxanthine, prepared as cocrystals using Pterostilbene, or Theophylline as cocrystals using Resveratrol, the bitterness of the Theacrine, Theobromine, Methylliberine, Paraxanthine, Caffeine, and Theophylline is completely reduced or eliminated.

In an embodiment, the present invention relates to cocrystals of Methylxanthines with Stilbenoids as coformers wherein said cocrystal exhibits a pleasant taste and is free from the bitterness of Methylxanthines.

The Methylxanthines are selected from Theacrine, Theobromine, Methylliberine, Paraxanthine, Caffeine, and Theophylline.

In an embodiment, the present invention relates to cocrystals, and their polymorphs derived from Methylxanthines-selected from Theacrine, Theobromine, Methylliberine, Paraxanthine, Caffeine, and Theophylline alone or mixtures thereof with coformers selected from Stilbenoids;
wherein said cocrystal exhibits a pleasant taste and is free from the bitterness of Methylxanthines.

The Stilbenoids are selected from Hydroxy derivatives of Styrenes, preferably Pterostilbene and Resveratrol.

In an embodiment, the present invention relates to cocrystals of Methylxanthines with Stilbenoids as coformers, and to the polymorphic forms thereof, comprising;
(a) Theacrine: Pterostilbene
(b) Theobromine: Pterostilbene
(c) Methylliberine: Pterostilbene
(d) Paraxanthine: Pterostilbene
(e) Caffeine: Pterostilbene and
(f) Theophylline: Resveratrol
wherein said cocrystal exhibits a pleasant taste and is free from the bitterness of Methylxanthines.

The Methylxanthines and the co-formers present in the cocrystal are in a molar ratio of 1:4 to 4:1, particularly in the ratio 1:1.

In an embodiment, the cocrystal of Theacrine: Pterostilbene (1:1) is characterized by ¹HNMR (400 MHz, dmso-d6): δ 3.19 (3H, s), 3.39 (3H, s), 3.52 (3H, s), 3.63 (3H, s), 3.76 (6H, s), 6.36 (1H, s), 6.70 (2H, s), 6.76 (2H, d, J=8.4 Hz), 6.93 (1H, d, J=16.4 Hz), 7.15 (1H, d, J=16.4 Hz), 7.41 (2H, d, J=8.4 Hz), 9.59 (1H, s).

In another embodiment, the cocrystal of Theobromine: Pterostilbene (1:1) is characterized by ¹HNMR (400 MHz, dmso-d6): δ 3.33 (3H, s), 3.76 (6H, s), 3.84 (3H, s), 6.36 (1H, t, J=2.4 Hz), 6.71 (2H, t, J=2.4 Hz), 6.77 (2H, d, J=8.8 Hz), 6.94 (1H, d, J=16.4 Hz), 7.15 (1H, d, J=16.4 Hz), 7.41 (2H, d, J=8.8 Hz), 7.97 (1H, s), 9.60 (1H, s), 11.12 (1H, s).

The cocrystal of Methylliberine: Pterostilbene (1:1) characterized by ¹HNMR (400 MHz, dmso-d6): δ 3.21 (3H, s), 3.31 (3H, s), 3.40 (3H, s), 3.76 (6H, s), 4.01 (3H, s), 6.36 (1H, t, J=2 Hz), 6.71 (2H, d, J=1.6 Hz), 6.76 (2H, d, J=8.8 Hz), 6.93 (1H, d, J=16.8 Hz), 7.15 (1H, d, J=16.8 Hz), 7.41 (2H, d, J=8.8 Hz), 9.59 (1H, s).

The cocrystal of Caffeine: Pterostilbene (1:1) characterized by ¹HNMR (400 MHz, DMSO-d6): δ 3.20 (3H, s), 3.40 (3H, s), 3.76 (6H, s), 3.86 (3H, s), 6.36 (1H, t, J=2.0 Hz), 6.71 (2H, d, J=2.0 Hz), 6.76 (2H, d, J=8.8 Hz), 6.93 (1H, d, J=16.4 Hz), 7.15 (1H, d, J=16.4 Hz), 7.41 (2H, d, J=8.4 Hz), 7.99 (1H, s), 9.59 (1H, s).

The cocrystal of Paraxanthine: Pterostilbene (1:1) characterized by ¹HNMR (400 MHz, DMSO-d6): δ 3.17 (3H, s), 3.76 (6H, s), 3.85 (3H, s), 6.36 (1H, s), 6.71 (2H, s), 6.76 (2H, d, J=8.4 Hz), 6.94 (1H, d, J=16.8 Hz), 7.15 (1H, d, J=16.4 Hz), 7.41 (2H, d, J=8.0 Hz), 7.92 (1H, s), 9.59 (1H, s), 11.84 (1H, s).

The cocrystal of Theophylline: Resveratrol (1:1) characterized by ¹HNMR (400 MHz, dmso-d6): δ 3.24 (3H, s), 3.44 (3H, s), 6.12 (1H, t, J=2 Hz), 6.39 (2H, d, J=2 Hz), 6.75 (2H, d, J=8.4 Hz), 6.81 (1H, d, J=16.4 Hz), 6.92 (1H, d, J=16.4 Hz), 7.38 (2H, d, J=8.4 Hz), 8.04, (1H, s) 9.21 (2H, s), 9.54 (1H, s), 13.57 (1H, s).

In accordance with the same, the invention provides a process for debittering of Methylxanthines which process comprises, co-crystallization of Methylxanthines either with Pterostilbene or with Resveratrol, in 1:4 to 4:1 ratio preferably 1:1 ratio.

The co-crystallization process may be carried out by using with or without solvent.

In an aspect, the present invention provides a process for the preparation of cocrystals and their polymorphs of Methylxanthines with coformers comprising;
(a) Dissolving the coformer in a solvent at 35-55° C. to obtain clear solution followed by addition of Methylxanthines;
(b) Stirring the above mixture at a temperature of 40-60° C. to obtain a clear solution;
(c) Gradually cooling the above solution to ambient temperature and maintaining said temperature for a period of 10-20 h to allowing the co-crystallization; and
(d) Separating the co-crystal thus formed on standing followed by filtering, washing and drying.

The solvent for the process is selected from water, C1-C5 alcohols, lower organic acids such as formic acid, acetic acid, propanoic acid, butyric acid, ethers, esters, ketones alone or mixtures thereof.

In an alternate embodiment, the process for the preparation of debittering Methylxanthines comprises mechanical grinding a mixture of methylxanthines and Stilbenoids in a molar ratio of 1:4 to 4:1 preferably 1; 1 ratio.

In another embodiment, the present invention provides a process for masking the bitterness of Methylxanthines comprising;
(a) Dissolving the Methylxanthine selected from Theacrine, Theobromine, Methylliberine, Caffeine, Paraxanthine or Theophylline in suitable solvent or mixture of solvents followed by adding the co-former selected from Pterostilbene or Resveratrol in the molar ratio 1:4 to 4:1 preferably 1:1
(b) Allowing the mixture to be stirred until a clear solution is obtained;
(c) Separating the co-crystal thus formed on standing the solution of step (a) followed by filtering, washing and drying to obtain desired cocrystal.

In yet another embodiment, the method of masking the bitterness of Methylxanthines selected from Theacrine, Theobromine, Methylliberine, Caffeine, Paraxanthine or Theophylline comprises mechanical grinding of a mixture of methylxanthines and Pterostilbene or Resveratrol in a molar ratio of 1:4 to 4:1 preferably 1:1 ratio to obtain the desired cocrystal.

Accordingly, in an embodiment, the invention relates to a process for masking the bitterness of Theacrine, by the formation of cocrystal with Pterostilbene as a coformer, which involves dissolving Theacrine with Pterostilbene in a molar ratio of 1:4 to 4:1, preferably 1:1 ratio, in a suitable solvent. In an alternate embodiment, the invention discloses a process for masking the bitterness of Theacrine, by the formation of cocrystal with Pterostilbene as a coformer, which comprises mechanical grinding of Theacrine and Pterostilbene in the molar ratio of 1:4 to 4:1, preferably 1:1 ratio.

In another embodiment, the invention discloses a process for masking the bitterness of Theobromine, by the formation of cocrystal with Pterostilbene as a coformer, which involves dissolving Theobromine with Pterostilbene in a molar ratio of 1:4 to 4:1, preferably 1:1 ratio, in a suitable solvent. In an alternate embodiment, the invention relates to a process for masking the bitterness of Theobromine, by the formation of cocrystal with Pterostilbene as a coformer, which comprises mechanical grinding of Theobromine and Pterostilbene in the molar ratio of 1:4 to 4:1, preferably 1:1 ratio.

In yet another embodiment, the invention discloses a process for masking the bitterness of Methylliberine, by the formation of cocrystal with Pterostilbene as a coformer, which involves dissolving Methylliberine with Pterostilbene in a molar ratio of 1:4 to 4:1, preferably 1:1 ratio, in a suitable solvent. In an alternate embodiment, the invention discloses a process for masking the bitterness of Methylliberine, by the formation of cocrystal with Pterostilbene as a coformer, which comprises mechanical grinding of Methylliberine and Pterostilbene in the molar ratio of 1:4 to 4:1, preferably 1:1 ratio.

In yet another embodiment, the invention relates to a process for masking the bitterness of Caffeine, by the formation of a novel polymorphs of cocrystals with Pterostilbene as a coformer, which involves dissolving Caffeine with Pterostilbene in a molar ratio of 1:4 to 4:1, preferably 1:1. in a suitable solvent. In an alternate embodiment, the invention provides a process for masking the bitterness of Caffeine, by the formation of cocrystal with Pterostilbene as a coformer, which comprises the mechanical grinding of Caffeine and Pterostilbene in the molar ratio of 1:4 to 4:1, preferably 1:1 ratio.

In a further embodiment, the invention relates to a process for masking the bitterness of Paraxanthine, by the formation of cocrystal with Pterostilbene as a coformer, which involves dissolving Paraxanthine with Pterostilbene in a molar ratio of 1:4 to 4:1, preferably 1:1 ratio, in a suitable solvent. In an alternate embodiment, the invention discloses a process for masking the bitterness of Paraxanthine, by the formation of cocrystal with Pterostilbene as a coformer, which comprises mechanical grinding of Paraxanthine and Pterostilbene in the molar ratio of 1:4 to 4:1, preferably 1:1 ratio.

In yet another embodiment, the invention relates to a process for masking the bitterness of Theophylline, by the formation of cocrystal with Resveratrol as a coformer, which involves dissolving Theophylline with Resveratrol in a molar ratio of 1:4 to 4:1, preferably 1:1 ratio, in a suitable solvent. In an alternate embodiment, the invention discloses a process for masking the bitterness of Theophylline, by the formation of cocrystal with Resveratrol as a coformer, which comprises mechanical grinding of Theophylline with Resveratrol in the molar ratio of 1:4 to 4:1, preferably 1:1 ratio.

The solvent for the process is selected from C1-C5 alcohols; water; acids such as formic acid, acetic acid, propanoic acid, butyric acid; ethers, esters, ketones and the like alone or mixtures thereof

In a further embodiment, the mechanical grinding is carried out in presence of a solvent or in the absence of a solvent. When solvent is used the same is selected from C1-C5 alcohols; water; acids such as formic acid, acetic acid, propanoic acid, butyric acid; ethers, esters, ketones and the like alone or mixtures thereof.

In yet another embodiment, the Theacrine, Theobromine, Methylliberine, Paraxanthine, Caffeine, Theophylline, Pterostilbene and Resveratrol used in the present invention are chemically synthesized compounds.

In yet another embodiment, the invention provides novel cocrystal of Theacrine with Pterostilbene.

In another embodiment, the invention provides process for the preparation of cocrystals of Theacrine with Pterostilbene in a ratio of 1:1 and various polymorphic forms thereof.

In additional embodiments, the invention provides Theacrine and Pterostilbene cocrystals polymorphic Form I; Form II; Form III and Form IV, V and processes for preparation thereof.

Accordingly, the invention provides crystalline Theacrine: Pterostilbene cocrystal polymorphic Form I, which is characterized by an X-ray powder diffraction pattern having one or more peaks with relative intensity at about 12.78 (100%), 14.18 (90.2%), 14.69 (17.9%), 15.55 (19.0%), 17.45 (11.8%), 19.04 (9.1%), 22.28 (12.0%), 23.46 (14.4%), 27.90 (5.0%), 28.80 (5.6%), 29.50 (7.3%), 31.50 (7.6%), 33.04 (10.1%), 38.90 (14.2%), 41.02 (6.1%), 41.93 (4.7%), 53.16 (4.5%), 72.73 (8.4%)±0.2° 2θ

In another embodiment, the invention discloses crystalline Theacrine: Pterostilbene cocrystal polymorphic Form II, which is characterized by an X-ray powder diffraction pattern having one or more peaks with relative intensity at about 10.98 (4.8%), 13.06 (33.6%), 14.45 (65.4%), 15.09 (38.5%), 15.85 (32.3%), 17.71 (6.5%), 19.18 (13.9%), 20.21 (28.7%), 21.44 (6.8%), 22.58 (11.1%), 23.73 (9.5%), 26.40 (59.7%), 27.21 (100%), 28.29 (10.0%), 29.12 (2.7%), 29.87 (3.6%), 31.76 (5.8%), 32.10 (6.2%), 33.33 (6.1%), 35.73 (1%), 37.02 (1.5%), 39.20 (9.0%), 40.66 (1.0%), 45.00 (1.4%), 46.38 (1.8%), 47.41 (1.2%), 48.05 (1.7%), 54.61 (2.8%), 55.46 (2.4%) and 72.69 (2.5%)±0.2° 2θ.

In yet another embodiment, the invention relates to a crystalline Theacrine: Pterostilbene cocrystal polymorphic Form III, which is characterized by an X-ray powder diffraction pattern having one or more peaks with relative intensity at about 12.90 (64.2%), 14.32 (100%), 15.64 (13.3%), 17.45 (8.2%), 19.17 (6.6%), 21.69 (10.5%), 22.38 (7.9%), 23.58 (10.0%), 25.07 (7.0%), 26.19 (6.1%), 26.98 (9.6%), 28.86 (1.9%), 29.59 (2.8%), 31.58 (2.7%), 33.12 (3.7%), 38.98 (7%), 41.10 (1.4%), 43.62 (1.1%), 47.80 (1.0%), and 72.70 (2.7%)±0.2° 2θ.

According to another embodiment, the invention discloses crystalline Theacrine: Pterostilbene cocrystal polymorphic Form IV, which is characterized by an X-ray powder diffraction pattern having one or more peaks with relative intensity at about 12.98 (100%), 14.38 (97.8%), 15.72 (18.6%), 17.66 (14.9%), 19.38 (10.3), 21.72 (7.5%), 22.47 (9.1%), 23.69 (12.8%), 25.14 (8.1%), 26.16 (11.6%), 27.00 (17.2%), 28.03 (2.5%), 29.69 (4.4%), 30.83 (1.1%), 31.66 (4.3%), 33.25 (7.2%), 36.88 (1.1%), 39.12 (11.6%), 41.21 (2.1%), 43.62 (1.3%), 44.06 (1.7%), 44.83 (1.3%), 45.99 (1.0%), 47.30 (1.1%), 47.95 (1.9%), 49.04 (1.0%), 53.36 (1.5%), 72.68 (3.3%) and 88.31 (1.0%)±0.2° 2θ.

In another embodiment, the invention relates to a crystalline Theacrine: Pterostilbene cocrystal polymorphic Form V, which is characterized by an X-ray powder diffraction pattern having one or more peaks with relative intensity at about 12.83 (17.0%), 14.33 (100%), 15.72 (18.8%), 19.15 (3.5%), 20.06 (3.5%), 22.10 (8.8%), 23.55 (6.5%), 27.17 (5.8%), 31.73 (4.8%), 33.71 (2.0%), 37.88 (1.4%), 39.19 (6.2%), 41.07 (1.4%), 41.54 (2.2%), 43.86 (1.0%)±0.2° 2θ.

In another embodiment, the invention relates to a crystalline Theobromine: Pterostilbene cocrystal, which is characterized by an X-ray powder diffraction pattern having one or more peaks with relative intensity at about 11.75 (30.21%), 13.38 (63.19%), 15.37 (7.89%), 17.31 (10.18%), 19.35 (95.4%), 23.46 (44.32%), 26.98 (100%), 29.35 (14.13%), 31.65 (6.47%), 34.30 (2.19%), 35.93 (6.2%), 39.15 (5.4%), 40.67 (3.61%), 42.08 (3.2%), 44.61 (2.87%), 48.89 (2.4%), 50.06 (2.9%), 52.53 (2.36%), 55.83 (2.22%), 72.73 (6.96%), 88.42 (2.35%)±0.2° 2θ.

In a further embodiment, the invention provides novel cocrystal of Methylliberine with Pterostilbene. Accordingly, in a further embodiment, the invention provides Methylliberine and Pterostilbene cocrystals polymorphic Form 1, Form II, Form III, and Form IV and processes for the preparation thereof.

According to another aspect, the invention provides crystalline Methylliberine: Pterostilbene cocrystal polymorphic Form I, which is characterized by an X-ray powder diffraction pattern having one or more peaks at about 12.25 (87.7%), 13.33 (100%), 15.52 (24.8%), 16.44 (6.6%), 17.98 (6.0%), 19.99 (16.5%), 21.12 (8.6%), 21.99 (5.2%), 23.93 (4.5%), 25.44 (6.6%), 27.05 (11.1%), 28.76 (3.9%), 34.75 (3.8%), 37.81 (5.8%), 40.49 (3.0%), 42.78 (4.9%), 44.64 (3.9%), 72.73 (5.8%)±0.2° 2θ

In another embodiment, the invention discloses crystalline Methylliberine: Pterostilbene cocrystal polymorphic Form II, which is characterized by an X-ray powder diffraction pattern having one or more peaks with relative intensity at about 12.43 (68.3%), 13.49 (100%), 15.65 (11.9%), 16.60 (11%), 18.12 (4.6%), 20.13 (15.3%), 21.30 (7.2%), 22.16 (6.2%), 23.34 (1.5%), 24.08 (4.4%), 25.69 (17%), 26.91 (16.8%), 27.31 (22.4%), 29.91 (1.3%), 30.82 (1.2%), 31.29 (1.6%), 34.51 (1.3%), 36.24 (1.4%), 37.94 (3.7%), 42.82 (1.0%), 44.67 (1.1%), 72.70 (2.5%)±0.2° 2θ.

In another embodiment, the invention discloses crystalline Methylliberine: Pterostilbene cocrystal polymorphic Form III, which is characterized by an X-ray powder diffraction pattern having one or more peaks with relative intensity at about 13.20 (100%), 15.35 (16.1%), 16.50 (11.8%), 17.90 (3.2%), 19.88 (13.3%), 20.83 (3.9%), 22.06 (5.0%), 23.05 (1.3%), 23.88 (4.1%), 25.60 (14.9%), 36.03 (1.1%), 42.05 (1.2%), and 72.67 (7.7%)±0.2° 2θ.

In another embodiment, the invention discloses crystalline Methylliberine: Pterostilbene cocrystal polymorphic Form IV, which is characterized by an X-ray powder diffraction pattern having one or more peaks with relative intensity at about 12.00 (43.3%), 12.48 (91.5%), 13.63 (84.8%), 15.32 (11.0%), 16.77 (24.7%), 18.34 (10.8%), 19.02 (11.1%), 20.22 (20.4%), 21.44 (16.9%), 22.32 (14.8%), 23.34 (3.9%), 24.24 (10.5%), 25.84 (100%), 27.48 (81.2%), 33.31 (2.7%), 34.78 (5.1%), 35.01 (4.9%), 36.44 (3.5%), 38.10 (8.2%), 40.78 (1.5%), 43.12 (4.9%), 46.52 (2.7%), 47.75 (1.7%), 49.85 (1.4%)±0.2° 2θ.

In additional embodiments, the invention discloses Novel polymorphic forms of Caffeine and Pterostilbene cocrystals Form 1, Form II, Form III, and Form IV and processes for the preparation thereof.

The polymorphic forms of the cocrystals of Methylxanthines of the present invention are functionally similar to said cocrytal and mask the bitterness of methylxanthines.

Accordingly, the invention relates to crystalline Caffeine: Pterostilbene cocrystal polymorphic Form I, which is characterized by an X-ray powder diffraction pattern having one or more peaks with relative intensity at about 11.90 (28.6%), 15.03 (100%), 16.81 (51.2%), 19.06 (53.5%), 21.65 (44.4%), 25.81 (94.2%), 26.35 (91.04%), 28.16 (10.4%), 29.54 (6.7%), 31.32 (4.8%), 32.52 (10.3%), 35.74 (3.6%), 36.99 (3.8%), 39.36 (3.7%), 40.93 (7.3%), 43.66 (5.7%), 45.45 (3.6%), 49.83 (2.1%), 72.74 (8.5%) and 88.35 (3.3%)±0.2° 2θ,

In another embodiment, the invention discloses crystalline Caffeine: Pterostilbene cocrystal polymorphic Form II, which is characterized by an X-ray powder diffraction pattern having one or more peaks with relative intensity at about 12.83 (2.19%), 17.04 (5.43%), 19.41 (5.24%), 22.25 (6.28%), 26.31 (100%), 28.67 (3.8%), 30.04 (1.6%), 32.96 (2.3%), 41.34 (1.1%), and 53.94 (1.7%)±0.2° 2θ,

In another embodiment, the invention discloses crystalline Caffeine: Pterostilbene cocrystal polymorphic Form III, which is characterized by an X-ray powder diffraction pattern having one or more peaks with relative intensity at about 10.53 (18.3%), 12.18 (4.9%), 14.01 (23.04%), 15.56 (25.3%), 17.22 (21.0%), 19.35 (16.7%), 22.13 (13.7%), 24.02 (4.3%), 26.51 (100%), 28.57 (20.4%), 30.22 (4.0%), 31.82 (4.1%), 36.23 (1.1%), 43.3 (1.8%), 46.52 (1.1%), 53.52 (1.1%) and 72.72 (2.2%)±0.2° 2θ

According to another aspect, the invention provides crystalline Caffeine: Pterostilbene cocrystal polymorphic Form IV, which is characterized by an X-ray powder diffraction pattern having one or more peaks with relative intensity at about 10.49 (100%), 12.24 (18.5%), 13.92 (35.3%), 14.18 (53.8%), 15.49 (41.5%), 15.60 (63.8%), 17.08 (36.3%), 19.3 (24.9%), 19.5 (60.2%), 22.13 (35.6%), 23.77 (12.7%), 26.06 (79.5%), 26.7 (94.8%), 28.51 (17.7%), 30.00 (13.0%), 32.48 (10.0%), 36.36 (10.3%), 38.15 (12.0%), 40.00 (8.1%), 41.28 (10.4%), 43.26 (7.5%), 46.34 (14.8%), 50.19 (8.5%), 53.95 (9.2%), 56.00 (5.7%)±0.2° 2θ

In another embodiment, the invention discloses novel cocrystals of Paraxanthine with Pterostilbene.

Accordingly, the cocrystals of Paraxanthine with Pterostilbene in a ratio of 1:1 is characterized by an X-ray powder diffraction pattern having one or more peaks with relative intensity at about 11.31 (12.8%), 15.88 (71.8%), 19.86 (18.7%), 21.28 (26.0%), 22.52 (33.6%), 24.9 (83.2%), 26.96 (100%), 29.00 (12.3%), 29.21 (19.3%), 30.41 (14.3%), 32.45 (9.9%), 38.8 (6.5%), 40.68 (5.2%), 44.84 (4.9%), 50.55 (3.6%), 55.48 (4.4%)±0.2° 2θ

In another embodiment, the invention discloses novel cocrystals of Theophylline with Resveratrol. Accordingly, the cocrystals of Theophylline with Resveratrol in a ratio of 1:1 is characterized by an X-ray powder diffraction pattern having one or more peaks with relative intensity at about 10.30 (9.4%), 12.40 (30.7%), 13.81 (100%), 15.21 (9.4%), 16.27 (14.1%), 17.21 (4.5%), 19.22 (21.1%), 20.76 (13.0%), 22.30 (8.4%), 23.52 (8.4%), 24.84 (47.9%), 25.36 (33.6%), 26.64 (7.4%), 28.26 (8.6%), 30.68 (8.9%), 32.88 (3.3%), 34.96 (1.1%), 36.46 (1.5%), 37.61 (1.1%), 38.45 (2.0%), 39.84 (1.4%), 42.16 (2.3%), 45.91 (1.3%), 52.26 (1.3%), 72.73 (4.1%) and 88.35 (1.2%)±0.2° 2θ.

These polymorphic forms are reliably characterized by peak positions in the X-ray diffractogram, which produces a fingerprint of the crystalline form and is able to distinguish it from all other crystalline and amorphous forms of Theacrine or Methylliberine or Theobromine or Caffeine or Paraxanthine with Pterostilbene. Measurements of 20 values are accurate to within ±0.2 degrees. All the powder diffraction patterns were measured on a PANalytical X'Pert3 X-ray powder diffractometer with a copper-K-a radiation source.

In an embodiment, the present invention discloses a composition comprising an effective amount of the cocrystal or its polymorphs of Methylxanthines selected from Theacrine, Theobromine, Methylliberine, Caffeine, Paraxanthine and Theophylline with coformers selected from Stilbenoids and one or more excipients.

The excipients are selected from one or more of a binder, filler, lubricant, emulsifier, suspending agent, sweetener, preservative, buffer, wetting agent, disintegrant, diluents, flavours, colours, suitable polymers effervescent agent, additive, and mixtures thereof.

The additive is selected from the group consisting of microcrystalline cellulose, lactose, sucrose, fructose, glucose, dextrose, dibasic calcium phosphate, calcium sulfate, cellulose, methylcellulose, cellulose derivatives, kaolin, mannitol, lactitol, maltitol, xylitol, sorbitol, sugar alcohols, dry starch, dextrins, maltodextrin, polysaccharides, and mixtures thereof.

In yet another embodiment, the present invention relates to a nutraceutical or pharmaceutical dosage form comprising cocrystal or its polymorphs of Methylxanthines selected from Theacrine, Theobromine, Methylliberine, Caffeine, Paraxanthine and Theophylline with coformers selected from Stilbenoids.

In another embodiment, the present invention relates to the pharmaceutical composition comprising an effective amount of the cocrystal or its polymorphs of Methylxanthines selected from Theacrine, Theobromine, Methylliberine, Caffeine, Paraxanthine and Theophylline with coformers selected from Stilbenoids and one or more excipients for dermatological use.

The nutraceutical or pharmaceutical dosage wherein the dosage form is an oral dosage form selected from the group consisting of a tablet, capsule, powder, suspension and lozenge.

The coformers selected from Pterostilbene or Resveratrol used in the present invention masks the bitterness of the Methylxanthines by blocking the sensory receptors responsible for taste.

The taste is the realization of sensation when a nutraceutical or a medicine is placed in the oral cavity. Preference tests provide information about whether testers like or dislike a product. The taste assessment trials are designed by using statistical tools to minimize or reduce the bias in responses within and between human volunteers. For sensory analysis one of the important part is to test the taste buds of healthy volunteers.

Smell, taste and texture are the three main components affecting the palatability of a product. Palatability is referred to as the voluntary acceptance and consumption of a pharmaceutical composition which can be measured by acceptance, preference and consumption tests and when a substance has a 90% or higher voluntary acceptance rate in the test subjects; the product is deemed to be palatable.

Accordingly, round table evaluations were performed in six alternate days with a set of eight panelists. Water and unsalted crackers were asked to be consumed by the panelists to clear their palates before testing.

On day one Theacrine and equal concentrations (amounts) of Theacrine: Pterostilbene co-crystals were taken by all the panelists for sensorial analysis.

On day two Theobromine and equal concentrations (amounts) of Theobromine: Pterostilbene co-crystals were taken by all the panelists for sensorial analysis.

On Day three Methylliberine and equal concentrations (amounts) of Methylliberine: Pterostilbene co-crystals were taken for sensorial analysis, by all the panelists.

On Day four Caffeine and equal concentrations (amounts) of Caffeine: Pterostilbene cocrystal were taken for sensorial analysis, by all the panelists.

On Day five Paraxanthine and equal concentrations (amounts) of Paraxanthine: Pterostilbene cocrystal were taken for sensorial analysis, by all the panelists.

On Day six Theophylline and equal concentrations (amounts) of Theophylline: Resveratrol cocrystal were taken for sensorial analysis, by all the panelists.

Results were recorded by individual panelists in respective forms indicating their preference (like) and non-preference (dislike) for consumption. The results of the sensorial testing showed clear preference by all the eight panelists for the cocrystals of the present invention over the pure Theacrine, Theobromine, Methylliberine, Caffeine, Paraxanthine, and Theophylline as the bitterness of Theacrine, Theobromine, Methylliberine, Caffeine, Paraxanthine, and Theophylline is completely masked in cocrystal forms. The results are provided in the table 1 below.

As is evident from the table below, a clear preference was shown by the panellist for co-crystallised methylxanthines compounds over the pure methylxanthines, which conclusively indicates that the Pterostilbene and Resveratrol debitters the methylxanthines by masking its bitter taste.

In a further embodiment, the co-crystal composition of the present invention produces variety of health benefits based on the dosage form such as improved memory, alertness, focus, relax, enhances energy and stamina.

Additionally, the crystal formers viz., Pterostilbene and Resveratrol being a safe and tested nutraceuticals and thus improved the nutraceutical effect of Theacrine, Theobromine, Methylliberine, Caffeine, Paraxanthine and Theophylline synergistically.

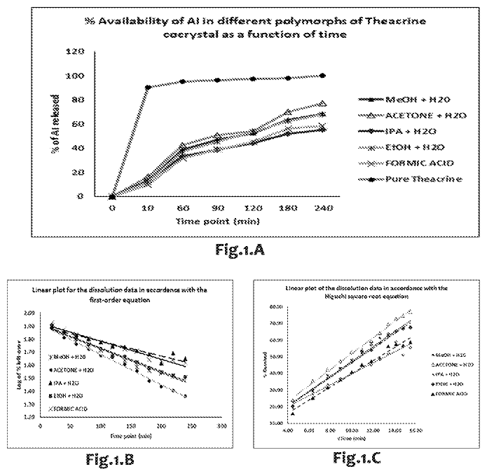

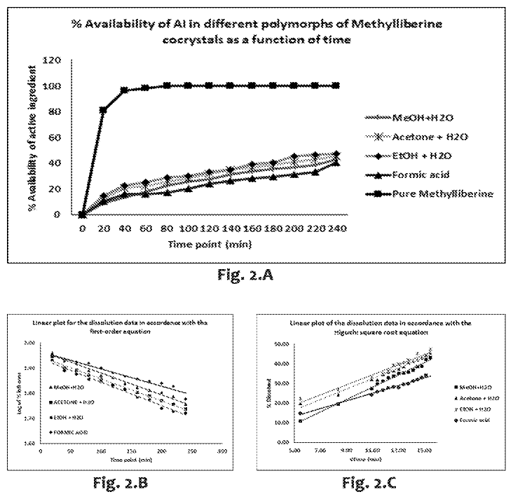

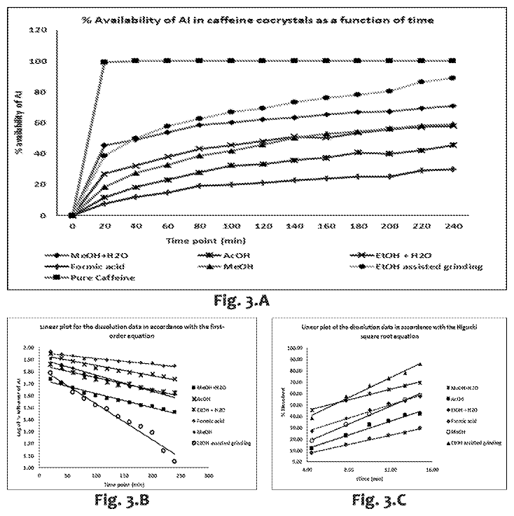

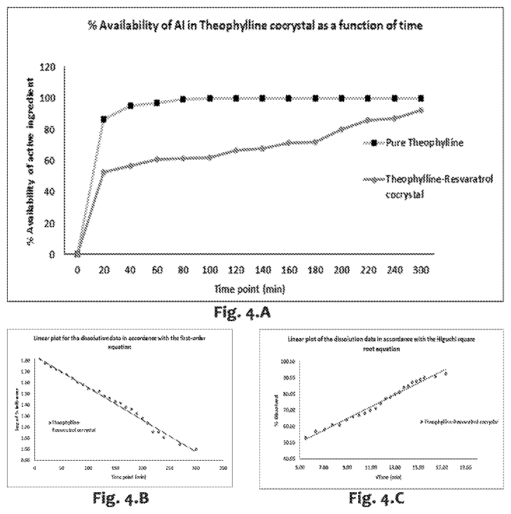

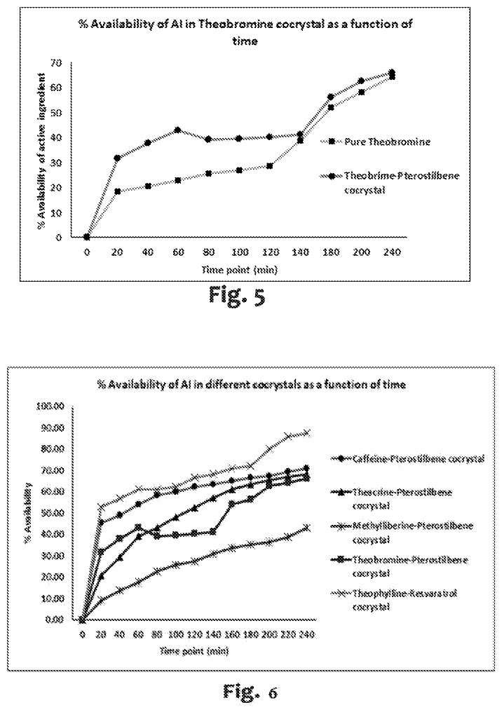

In an embodiment, the dissolution study of the cocrystals of Methylxanthines with the coformers Pterostilbene and Resveratrol were evaluated. It was observed that in all cases the dissolution rate did not follow the zero-order kinetics which means the dissolution rate is independent of the amount of availability of active ingredient. The cocrystals have lower solubility, slow-release rate and thereby more bioavailable when compared to their pure forms (FIG. 1 to 6).

### Experimental:

The following examples, which include preferred embodiments, will serve to illustrate the practice of this invention, it being understood that the particulars shown are by way of example and for the purpose of illustrative discussion of preferred embodiments of the invention.

### General Method for the Synthesis of Cocrystals:

The general synthetic method for the preparation of the novel cocrystals, which process comprises a coformer (Pterostilbene or Resveratrol) is added to the suitable solvent and dissolved at 45-50° C. to get clear solution followed by the addition of Methylxanthine (Theacrine, Theobromine, Methylliberine, Paraxanthine, Caffeine and Theophylline). Stirring was continued at 45-50° C. for 30 min to obtain clear solution. Reaction mass was gradually allowed to cool to ambient temperature and maintained at the same temperature for 12-18 h to crystalize out the corresponding cocrystals. The suitable solvent that can be employed for the synthesis of the cocrystals is selected from the group comprising C1-5 alcohols, water, formic acid, acetic acid, propanoic acid, butyric acid; ethers, esters, ketones and the like alone or mixtures thereof. The following additional techniques can also be used to prepare the Methylxanthine cocrystals of the invention; neat grinding, solvent drop grinding, reactive crystallization, anti-solvent addition, cooling crystallization and slow solvent evaporation.

### EXAMPLES

### Example-1: Preparation of Theacrine: Pterostilbene (1:1) Cocrystal (Form I)

To a stirred solution of Theacrine (5.0 g, 22.3 mmol) in a mixture of water (20 mL) and methanol (30 mL) at 65-70° C. was added Pterostilbene (5.72 g, 22.3 mmol) and the mixture stirred until it become clear solution. It was allowed to stand at rt for 24 h during which crystals were separated out. It was filtered, washed with water: methanol mixture (3 mL, 1:1.5) and dried in oven at 40-45° C. under vacuum. Yield: 9.82 g, mp: 143-145° C. ¹HNMR (400 MHz, dmso-d6): δ 3.19 (3H, s), 3.39 (3H, s), 3.52 (3H, s), 3.63 (3H, s), 3.76 (6H, s), 6.36 (1H, s), 6.70 (2H, s), 6.76 (2H, d, J=8.4 Hz), 6.93 (1H, d, J=16.4 Hz), 7.15 (1H, d, J=16.4 Hz), 7.41 (2H, d, J=8.4 Hz), 9.59 (1H, s). PXRD:12.78 (100%), 14.18 (90.2%), 14.69 (17.9%), 15.55 (19.0%), 17.45 (11.8%), 19.04 (9.1%), 22.28 (12.0%), 23.46 (14.4%), 27.90 (5.0%), 28.80 (5.6%), 29.50 (7.3%), 31.50 (7.6%), 33.04 (10.1%), 38.90 (14.2%), 41.02 (6.1%), 41.93 (4.7%), 53.16 (4.5%), 72.73 (8.4%)±0.2° 2θ. DSC: A sharp endotherm observed at 146.22° C.

### Example-2: Preparation of Theobromine: Pterostilbene (1:1) Cocrystal

Theobromine and Pterostilbene (1:1) were taken in a mortar and added acetic acid. The resultant mixture was grinded for 1 h with pestle. It was dried in oven at 40-45° C. under vacuum. ¹HNMR (400 MHz, dmso-d6): δ 3.33 (3H, s), 3.76 (6H, s), 3.84 (3H, s), 6.36 (1H, t, J=2.4 Hz), 6.71 (2H, t, J=2.4 Hz), 6.77 (2H, d, J=8.8 Hz), 6.94 (1H, d, J=16.4 Hz), 7.15 (1H, d, J=16.4 Hz), 7.41 (2H, d, J=8.8 Hz), 7.97 (1H, s), 9.60 (1H, s), 11.12 (1H, s). PXRD:11.75 (30.20%), 13.38 (63.10%), 15.37 (7.80%), 17.31 (10.10%), 19.35 (95.40%), 23.46 (44.30%), 26.98 (100%), 29.35 (14.10%), 31.65 (6.40%), 34.30 (2.10%), 35.93 (6.20%), 39.15 (5.40%), 40.67 (3.60%), 42.08 (3.20%), 44.61 (2.80%), 48.89 (2.40%), 50.06 (2.90%), 52.53 (2.3%), 55.83 (2.2%), 72.73 (6.90%), 88.42 (2.30%)±0.2° 2θ. DSC: A sharp endotherm observed at 93° C.

### Example-3: Preparation of Methylliberine: Pterostilbene (1:1) Cocrystal (Form I)

To a stirred solution of Methylliberine (5.80 g, 25.9 mmol) in a mixture of water (10 mL) and methanol (30 mL) at 65-70° C., was added Pterostilbene (6.63 g, 25.9 mmol) and the mixture stirred at the same temperature until it becomes a clear solution. It was brought to rt and allowed to stand at rt for 24 h during which crystals were separated. The crystals thus formed were filtered, washed with water: methanol mixture (3 mL, 1:3) and dried in oven at 40-45° C. under vacuum. Yield: 11.8 g, mp: 130-132° C. ¹HNMR (400 MHz, dmso-d6): δ 3.21 (3H, s), 3.31 (3H, s), 3.40 (3H, s), 3.76 (6H, s), 4.01 (3H, s), 6.36 (1H, t, J=2 Hz), 6.71 (2H, d, J=1.6 Hz), 6.76 (2H, d, J=8.8 Hz), 6.93 (1H, d, J=16.8 Hz), 7.15 (1H, d, J=16.8 Hz), 7.41 (2H, d, J=8.8 Hz), 9.59 (1H, s). PXRD: 12.25 (87.7%), 13.33 (100%), 15.52 (24.8%), 16.44 (6.6%), 17.98 (6.0%), 19.99 (16.5%), 21.12 (8.6%), 21.99 (5.2%), 23.93 (4.5%), 25.44 (6.6%), 27.05 (11.1%), 28.76 (3.9%), 34.75 (3.8%), 37.81 (5.8%), 40.49 (3.0%), 42.78 (4.9%), 44.64 (3.9%), 72.73 (5.8%)±0.2° DSC: A sharp endotherm observed at 132.54° C.

### Example-4: Preparation of Theophylline: Resveratrol (1:1) Cocrystal

To a stirred solution of Theophylline (0.5 g, 2.8 mmol) in a mixture of water (2 mL) and methanol (2 mL), at 80-83° C. was added Resveratrol (0.63 g, 2.8 mmol) and the mixture stirred at the same temperature until it becomes clear. The clear solution was allowed to stand at rt for 24 h during which crystals were separated. It was filtered, washed with water: ethanol mixture (3 mL, (1:3) and dried in oven at 40-45° C. under vacuum. Yield: 0.79 g, mp: 201-216° C. ¹HNMR (400 MHz, dmso-d6): δ 3.24 (3H, s), 3.44 (3H, s), 6.12 (1H, t, J=2 Hz), 6.39 (2H, d, J=2 Hz), 6.75 (2H, d, J=8.4 Hz), 6.81 (1H, d, J=16.4 Hz), 6.92 (1H, d, J=16.4 Hz), 7.38 (2H, d, J=8.4 Hz), 8.04 (1H, s) 9.21 (2H, s), 9.54 (1H, s), 13.57 (1H, s). PXRD: 10.30 (9.4%), 12.40 (30.7%), 13.81 (100%), 15.21 (9.4%), 16.27 (14.1%), 17.21 (4.5%), 19.22 (21.1%), 20.76 (13.0%), 22.30 (8.4%), 23.52 (8.4%), 24.84 (47.9%), 25.36 (33.6%), 26.64 (7.4%), 28.26 (8.6%), 30.68 (8.9%), 32.88 (3.3%), 34.96 (1.1%), 36.46 (1.5%), 37.61 (1.1%), 38.45 (2.0%), 39.84 (1.4%), 42.16 (2.3%), 45.91 (1.3%), 52.26 (1.3%), 72.73 (4.1%) and 88.35 (1.2%)±0.2° 2θ. A sharp endotherm observed at 201.95° C.

### Example 5: Preparation of Caffeine: Pterostilbene (1:1) Cocrystal (Form I)

To a stirred solution of caffeine (5.0 g, 25.7 mmol) in a mixture of water (10 mL) and methanol (30 mL) at 65-70° C. was added Pterostilbene (6.6 g, 25.7 mmol) and the mixture stirred until it become clear solution. It was allowed to stand at rt for 15 h during which crystals were separated out. It was filtered, washed with water: methanol mixture (3 mL, 1:1.5) and dried in oven at 40-45° C. under vacuum. Yield: 10.8 g, mp: 116-120° C. ¹HNMR (400 MHz, DMSO-d6): δ 3.20 (3H, s), 3.40 (3H, s), 3.76 (6H, s), 3.86 (3H, s), 6.36 (1H, t, J=2.0 Hz), 6.71 (2H, d, J=2.0 Hz), 6.76 (2H, d, J=8.8 Hz), 6.93 (1H, d, J=16.4 Hz), 7.15 (1H, d, J=16.4 Hz), 7.41 (2H, d, J=8.4 Hz), 7.99 (1H, s), 9.59 (1H, s). PXRD: 11.90 (28.6%), 15.03 (100%), 16.81 (51.2%), 19.06 (53.5%), 21.65 (44.4%), 25.81 (94.2%), 26.35 (91.04%), 28.16 (10.4%), 29.54 (6.7%), 31.32 (4.8%), 32.52 (10.3%), 35.74 (3.6%), 36.99 (3.8%), 39.36 (3.7%), 40.93 (7.3%), 43.66 (5.7%), 45.45 (3.6%), 49.83 (2.1%), 72.74 (8.5%) and 88.35 (3.3%)±0.2° 2θ, DSC: A sharp endotherm observed at 117.30° C.

### Example 6: Preparation of Paraxanthine: Pterostilbene (1:1) Cocrystal

Paraxanthine (70 mg, 0.388 mmol) and Pterostilbene (0.099 g, 0.388 mmol) were taken in a mortar and added acetic acid. The resultant mixture was ground for 1 h with pestle. It was dried in oven at 40-45° C. under vacuum. Yield (0.120 g), mp: 193.4-207.8° C. ¹HNMR (400 MHz, dmso-d6): δ ¹HNMR (400 MHz, DMSO-d6): δ 3.17 (3H, s), 3.76 (6H, s), 3.85 (3H, s), 6.36 (1H, s), 6.71 (2H, s), 6.76 (2H, d, J=8.4 Hz), 6.94 (1H, d, J=16.8 Hz), 7.15 (1H, d, J=16.4 Hz), 7.41 (2H, d, J=8.0 Hz), 7.92 (1H, s), 9.59 (1H, s), 11.84 (1H, s), PXRD: 11.31 (12.8%), 15.88 (71.8%), 19.86 (18.7%), 21.28 (26.0%), 22.52 (33.6%), 24.9 (83.2%), 26.96 (100%), 29.00 (12.3%), 29.21 (19.3%), 30.41 (14.3%), 32.45 (9.9%), 38.8 (6.5%), 40.68 (5.2%), 44.84 (4.9%), 50.55 (3.6%), 55.48 (4.4%) 0.2° 2θ. DSC: A sharp endotherm observed at 204.17° C.

### Polymorphs

### Example 7: Preparation of Theacrine and Pterostilbene Cocrystals: Polymorphic Form—II

To a stirred solution of Theacrine (2.0 g, 8.9 mmol) in a mixture of water (3 mL) and ethanol (10 mL) at 65-70° C. was added Pterostilbene (2.29 g, 8.9 mmol) and the mixture kept stirring until it becomes a clear solution. It was allowed to stand at rt for 24 h during which crystals were separated. It was filtered, washed with water: ethanol mixture (3 mL, 1:3) and dried in oven at 40-45° C. under vacuum. Yield: 3.5 g, mp: 144-146° C. PXRD: 10.98 (4.8%), 13.06 (33.6%), 14.45 (65.5%), 15.09 (38.6%), 15.85 (32.3%), 17.71 (6.6%), 19.18 (13.9%), 20.21 (28.7%), 21.44 (6.8%), 22.58 (11.1%), 23.73 (9.5%), 26.40 (59.7%), 27.21 (100%), 28.29 (10.1%), 29.12 (2.6%), 29.87 (3.6%), 31.76 (5.8%), 32.10 (6.3%), 33.33 (6.1%), 35.73 (1%), 37.02 (1.5%), 39.20 (9.0%), 40.66 (1.0%), 42.21 (0.9%), 45.00 (1.4%), 46.38 (1.8%), 47.41 (1.3%), 48.05 (1.8%), 53.41 (2.8%), 54.61 (2.5%), 55.46 (0.6%) and 72.69 (2.5%)±0.2° 2θ.

### Example 8: Preparation of Theacrine and Pterostilbene Cocrystals: Polymorphic Form—III

To a stirred solution of Theacrine (2.0 g, 8.9 mmol) in a mixture of water (4 mL) and acetone (4 mL) at 65-70° C. was added Pterostilbene (2.29 g, 8.9 mmol) and the mixture continued to be stirred until it becomes a clear solution. It was allowed to stand at rt for 24 h during which crystals were separated. It was filtered, washed with water: acetone mixture (3 mL, 1:1) and dried in oven at 40-45° C. under vacuum. Yield: 3.52 g, mp: 143-145° C. PXRD: 12.90 (64.3%), 14.32 (100%), 15.64 (13.3%), 17.45 (8.3%), 19.17 (6.7%), 21.69 (10.5%), 22.38 (7.9%), 23.58 (10.0%), 25.07 (7.1%), 26.19 (6.1%), 26.98 (9.6%), 27.93 (0.8), 28.86 (1.9%), 29.59 (2.8%), 31.58 (2.7%), 33.12 (3.7%), 38.98 (7%), 41.10 (1.4%), 43.62 (1.1%), 47.80 (1.0%), and 72.70 (2.7%)±0.2° 2θ.

### Example 9: Preparation of Theacrine and Pterostilbene Cocrystals: Polymorphic Form—IV

To a stirred solution of Theacrine (2.0 g, 8.9 mol) in a mixture of water (2 mL) and Isopropanol (5 mL) at 65-70° C. was added Pterostilbene (2.29 g, 8.9 mmol) and the mixture stirred until it becomes clear solution. It was allowed to stand at rt for 24 h during which crystals were separated. It was filtered, washed with water: isopropanol mixture (3 mL, 1:2.5) and dried in oven at 40-45° C. under vacuum. Yield: 4.05 g; mp: 143-145° C. PXRD:12.98 (100%), 14.38 (97.8%), 15.72 (18.6%), 17.66 (14.9%), 19.38 (10.3), 21.72 (7.5%), 22.47 (9.1%), 23.69 (12.8%), 25.14 (8.1%), 26.16 (11.6%), 27.00 (17.2%), 28.03 (2.5%), 29.69 (4.4%), 30.83 (1.1%), 31.66 (4.3%), 33.25 (7.2%), 36.88 (1.1%), 39.12 (11.6%), 41.21 (2.1%), 43.62 (1.3%), 44.06 (1.7%), 44.83 (1.3%), 45.99 (1.0%), 47.30 (1.2%), 47.95 (1.9%), 49.04 (1.0%), 53.36 (1.6%), 72.68 (3.4%) and 88.31 (1.0%)±0.2° 2θ.

### Example 10: Preparation of Theacrine and Pterostilbene Cocrystal: Polymorphic Form—V

To a stirred solution of Theacrine (2.0 g, 8.9 mol) in formic acid (3 mL) at 65-70° C. was added Pterostilbene (2.29 g, 8.9 mol) and the mixture continued to be stirred until it become clear solution. It was allowed to stand at rt for 24 h during which crystals separated. It was filtered and washed with water and dried in oven at 40-45° C. under vacuum. Yield: 2.8 g; mp: 143-146° C. PXRD: 12.83 (17.1%), 14.33 (100%), 15.72 (18.8%), 19.15 (3.6%), 20.06 (3.5%), 22.10 (8.8%), 23.55 (6.5%), 27.17 (5.9%), 31.73 (4.9%), 33.71 (2.0%), 37.88 (1.4%), 39.19 (6.2%), 41.07 (1.4%), 41.54 (2.2%), 43.86 (1.1%)±0.2° 2θ.

### Example 11: Preparation of Methylliberine and Pterostilbene Cocrystals: Polymorphic Form—II

To a stirred solution of Methylliberine (2.0 g, 8.9 mmol) in a mixture of water (3 mL) and ethanol (10 mL) at 65-70° C. was added Pterostilbene (2.29 g, 8.9 mmol) and the mixture stirred until it becomes clear solution. It was allowed to stand at rt for 24 h during which crystals were separated. It was filtered, washed water: methanol mixture with (3 mL, 1:3) and dried in oven at 40-45° C. under vacuum. Yield: 3.37 g; mp: 129-132° C. PXRD:12.43 (68.3%), 13.49 (100%), 15.65 (11.9%), 16.60 (11%), 18.12 (4.6%), 20.13 (15.4%), 21.30 (7.2%), 22.16 (6.3%), 23.34 (1.6%), 24.08 (4.4%), 25.69 (17.1%), 26.91 (16.9%), 27.31 (22.4%), 29.91 (1.4%), 30.82 (1.3%), 31.29 (1.6%), 34.51 (1.3%), 36.24 (1.4%), 37.94 (3.7%), 42.82 (1.0%), 44.67 (1.1%), 72.70 (2.5%)±0.2° 2θ.

### Example 12: Preparation of Methylliberine and Pterostilbene (1:1) Cocrystals: Polymorphic Form—III

To a stirred solution of Methylliberine (2.0 g, 8.9 mmol) in a mixture of water (4 mL) and Acetone (4 mL) at 65-70° C. was added Pterostilbene (2.29 g, 8.9 mmol) and the mixture stirred at this temperature until it becomes a clear solution. It was allowed to stand at rt for 24 h during which crystals were separated. It was filtered, washed water: methanol mixture with (3 mL, 1:3) and dried in oven at 40-45° C. under vacuum. Yield: 3.lg, mp: 130-133° C. PXRD: 13.20 (100%), 15.35 (16.1%), 16.50 (11.8%), 17.90 (3.3%), 19.88 (13.3%), 20.83 (3.9%), 22.06 (5.0%), 23.05 (1.3%), 23.88 (4.1%), 25.60 (14.9%), 36.03 (1.2%), 42.05 (1.2%), and 72.67 (7.7%)±0.2° 2θ.

### Example 13: Preparation of Methylliberine and Pterostilbene cocrystals: Polymorphic Form—IV

To a stirred solution of Methylliberine (2.0 g, 8.9 mmol) in formic acid (3 mL) at 65-70° C. was added Pterostilbene (2.29 g, 8.9 mmol) and the mixture stirred at the same temperature until it becomes a clear solution. It was allowed to stand at rt for 24 h during which crystals were separated. It was filtered, washed water: methanol mixture with (3 mL, 1:3) and dried in oven at 40-45° C. under vacuum. Yield: 3.84 g: mp:130-133° C. PXRD: 12.00 (43.4%), 12.48 (91.5%), 13.63 (84.8%), 15.32 (11.0%), 16.77 (24.7%), 18.34 (10.9%), 19.02 (11.1%), 20.22 (20.4%), 21.44 (16.9%), 22.32 (14.8%), 23.34 (3.9%), 24.24 (10.5%), 25.84 (100%), 27.48 (81.2%), 33.31 (2.7%), 34.78 (5.1%), 35.01 (4.9%), 36.44 (3.5%), 38.10 (8.2%), 40.78 (1.5%), 43.12 (4.9%), 46.52 (2.7%), 47.75 (1.7%), 49.85 (1.4%)±0.2° 2θ.

### Example 14: Preparation of Caffeine and Pterostilbene Cocrystals: Polymorphic Form—I

To a stirred solution of caffeine (5.0 g, 25.7 mmol) in a mixture of water (10 mL) and methanol (30 mL) at 65-70° C. was added Pterostilbene (6.6 g, 25.7 mmol) and the mixture stirred until it become clear solution. It was allowed to stand at rt for 15 h during which crystals were separated out. It was filtered, washed with water: methanol mixture (3 mL, 1:1.5) and dried in oven at 40-45° C. under vacuum. Yield: 10.8 g, mp: 116-120° C. PXRD:11.90 (28.6%), 15.03 (100%), 16.81 (51.2%), 19.06 (53.5%), 21.65 (44.4%), 25.81 (94.2%), 26.35 (91.04%), 28.16 (10.4%), 29.54 (6.7%), 31.32 (4.8%), 32.52 (10.3%), 35.74 (3.6%), 36.99 (3.8%), 39.36 (3.7%), 40.93 (7.3%), 43.66 (5.7%), 45.45 (3.6%), 49.83 (2.1%), 72.74 (8.5%) and 88.35 (3.3%)±0.2° 2θ.

### Example 15: Preparation of Caffeine and Pterostilbene Cocrystals: Polymorphic Form—II

To a stirred solution of caffeine (2.0 g, 10.29 mmol) in Formic acid (3 mL) at 75-80° C. was added Pterostilbene (2.63 g, 10.29 mmol) and the mixture stirred until it become clear solution. It was allowed to stand at rt for 15 h during which crystals were separated out. It was filtered, and dried in oven at 40-45° C. under vacuum. Yield: 3.2 g, mp: 116-127° C. PXRD:12.83 (2.19%), 17.04 (5.43%), 19.41 (5.24%), 22.25 (6.28%), 26.31 (100%), 28.67 (3.8%), 30.04 (1.6%), 32.96 (2.3%), 41.34 (1.1%), and 53.94 (1.7%)±0.2° 2θ.

### Example 16: Preparation of Caffeine and Pterostilbene Cocrystals: Polymorphic Form—III

To a stirred solution of caffeine (2.0 g, 10.29 mmol) in Acetic acid (4 mL) at 75-80° C. was added Pterostilbene (2.63 g, 10.29 mmol) and the mixture stirred until it become clear solution. It was allowed to stand at rt for 15 h during which crystals were separated out. It was filtered, and dried in oven at 40-45° C. under vacuum. Yield: 2.73 g, mp: 115-122° C. PXRD 10.53 (18.3%), 12.18 (4.9%), 14.01 (23.04%), 15.56 (25.3%), 17.22 (21.0%), 19.35 (16.7%), 22.13 (13.7%), 24.02 (4.3%), 26.51 (100%), 28.57 (20.4%), 30.22 (4.0%), 31.82 (4.1%), 36.23 (1.1%), 43.3 (1.8%), 46.52 (1.1%), 53.52 (1.1%) and 72.72 (2.2%)±0.2° 2θ.

### Example 17: Preparation of Caffeine and Pterostilbene Cocrystals: Polymorphic Form—IV

To a stirred solution of caffeine (2.0 g, 10.3 mmol) in a mixture of water (3 mL) and ethanol (10 mL) at 75-80° C. was added Pterostilbene (2.64 g, 10.3 mmol) and the mixture stirred until it become clear solution. It was allowed to stand at rt for 15 h during which crystals were separated out. It was filtered, washed with water: ethanol mixture (3 mL, 1:1.5) and dried in oven at 40-45° C. under vacuum. Yield: 3.9 g, mp: 119-122° C. PXRD 10.49 (100%), 12.24 (18.5%), 13.92 (35.3%), 14.18 (53.8%), 15.49 (41.5%), 15.60 (63.8%), 17.08 (36.3%), 19.3 (24.9%), 19.5 (60.2%), 22.13 (35.6%), 23.77 (12.7%), 26.06 (79.50), 26.7 (94.8%), 28.51 (17.7%), 30.00 (13.0%), 32.48 (10.0%), 36.36 (10.3%), 38.15 (12.0%), 40.00 (8.1%), 41.28 (10.4%), 43.26 (7.5%), 46.34 (14.8%), 50.19 (8.5%), 53.95 (9.2%), 56.00 (5.70)±0.2° 2θ.

### Example 18: Debittering Studies

Results were recorded by individual panellists in respective forms indicating their preference (like) and non-preference (dislike) for consumption which is tabulated below. The results of the sensorial testing showed clear preference by all the eight panellists for the cocrystals of the present invention over the pure Theacrine, Theobromine, Methylliberine, Caffeine, Paraxanthine, and Theophylline as the bitterness of Theacrine, Theobromine, Methylliberine, Caffeine, Paraxanthine, and Theophylline is completely masked in cocrystal forms.

| | TC | TB | ML | CA | PX | TP | TC:PB | TB:PB | ML:PB | CA:PB | PX:PB | TP:RS |
|---|---|---|---|---|---|---|---|---|---|---|---|---|
| | Prefer- | Prefer- | Prefer- | Prefer- | Prefer- | Prefer- | Prefer- | Prefer- | Prefer- | Prefer- | Prefer- | Prefer- |
| S. NO | ence | ence | ence | ence | ence | ence | ence | ence | ence | ence | ence | ence |
| Panelist- | No | No | No | No | No | No | Yes | Yes | Yes | Yes | Yes | Yes |
| 1 | | | | | | | | | | | | |
| Panelist- | No | No | No | No | No | No | Yes | Yes | Yes | Yes | Yes | Yes |
| 2 | | | | | | | | | | | | |
| Panelist- | No | No | No | No | No | No | Yes | Yes | Yes | Yes | Yes | Yes |
| 3 | | | | | | | | | | | | |
| Panelist- | No | No | No | No | No | No | Yes | Yes | Yes | Yes | Yes | Yes |
| 4 | | | | | | | | | | | | |
| Panelist- | No | No | No | No | No | No | Yes | Yes | Yes | Yes | Yes | Yes |
| 5 | | | | | | | | | | | | |
| Panelist- | No | No | No | No | No | No | Yes | Yes | Yes | Yes | Yes | Yes |
| 6 | | | | | | | | | | | | |
| Panelist- | No | No | No | No | No | No | Yes | Yes | Yes | Yes | Yes | Yes |
| 7 | | | | | | | | | | | | |
| Panelist- | No | No | No | No | No | No | Yes | Yes | Yes | Yes | Yes | Yes |
| 8 | | | | | | | | | | | | |

Note:
Theacrine (TC);
Theobromine (TB);
Methylliberine (ML);
Caffeine (CA),
Paraxanthine (PX)
Theophylline (TP);
Pterostilbene (PB);
Resveratrol (RS)

As is evident from the above, a clear preference is shown by the panellist for Cocrystal of methylxanthines compounds over the pure methylxanthines, which conclusively indicates that the conformers Pterostilbene and Resveratrol debitters the methylxanthines.

### Example 19: Dissolution Studies of Cocrystals

The dissolution data of various Methylxanthines cocrystals with Pterostilbene/Resveratrol systems are represented using 0^{th} order reaction rate, 1^{st} order reaction rate, and diffusion kinetics as depicted in FIG. 1 to FIG. 5. From the data, the dissolution pattern of the different cocrystal systems is summarized as follows:

### Zero-Order Kinetics:

In all cases, the curvilinear plot of % dissolved as a function of time indicates that the dissolution rate does not follow the zero-order kinetics which means the dissolution rate is independent on the amount of availability of active ingredient.

Solubility and release rate of active ingredient: From the below figures (1.A; 2.A; 3.A; and 4.A) it is evident that Methylxanthines viz Theacrine, Methylliberine, Caffeine, Paraxanthine and Theophylline in the form of cocrystals have lower solubility, slow-release rate and thereby more bioavailable when compared to their pure forms. In the case of Theobromine, cocrystal offered improved solubility leading to better bioavailability compared to pure Theobromine. Dissolution pattern of various cocrystal systems after a certain time in the graph is summarized below:

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1.-45. (canceled)

46. Cocrystals of Methylxanthine with Stilbenoid as coformer and polymorphic forms thereof.

47. The cocrystals as claimed in claim 46, wherein said cocrystal exhibits a pleasant taste and is free from the bitterness of Methylxanthine.

48. The cocrystals as claimed in claim 46, wherein the Methylxanthine is selected from Theacrine, Theobromine, Methylliberine, Caffeine, Paraxanthine or Theophylline and the like alone or mixtures thereof.

49. The cocrystals as claimed in claim 48, wherein the Stilbenoid is selected from Pterostilbene or Resveratrol.

50. The cocrystals as claimed in claim 49, wherein the Methylxanthine and Stilbenoid are in the ratio 1:4 to 4:1, preferably 1:1.

51. The cocrystals as claimed in claim 50, comprising:
a) Theacrine: Pterostilbene (1:1) characterized by ¹HNMR (400 MHz, dmso-d6): δ 3.19 (3H, s), 3.39 (3H, s), 3.52 (3H, s), 3.63 (3H, s), 3.76 (6H, s), 6.36 (1H, s), 6.70 (2H, s), 6.76 (2H, d, J=8.4 Hz), 6.93 (1H, d, J=16.4 Hz), 7.15 (1H, d, J=16.4 Hz), 7.41 (2H, d, J=8.4 Hz), 9.59 (1H, s); with DSC exhibiting a sharp endotherm at 146.22° C.;
b) Theobromine: Pterostilbene (1:1) characterized by ¹HNMR (400 MHz, dmso-d6): δ 3.33 (3H, s), 3.76 (6H, s), 3.84 (3H, s), 6.36 (1H, t, J=2.4 Hz), 6.71 (2H, t, J=2.4 Hz), 6.77 (2H, d, J=8.8 Hz), 6.94 (1H, d, J=16.4 Hz), 7.15 (1H, d, J=16.4 Hz), 7.41 (2H, d, J=8.8 Hz), 7.97 (1H, s), 9.60 (1H, s), 11.12 (1H, s); with DSC exhibiting a sharp endotherm at 93.0° C.;
c) Methylliberine: Pterostilbene (1:1) characterized by ¹HNMR (400 MHz, dmso-d6): δ 3.21 (3H, s), 3.31 (3H, s), 3.40 (3H, s), 3.76 (6H, s), 4.01 (3H, s), 6.36 (1H, t, J=2 Hz), 6.71 (2H, d, J=1.6 Hz), 6.76 (2H, d, J=8.8 Hz), 6.93 (1H, d, J=16.8 Hz), 7.15 (1H, d, J=16.8 Hz), 7.41 (2H, d, J=8.8 Hz), 9.59 (1H, s); with DSC exhibiting a sharp endotherm at 132.54° C.;
d) Caffeine: Pterostilbene (1:1) characterized by ¹HNMR (400 MHz, DMSO-d6): δ 3.20 (3H, s), 3.40 (3H, s), 3.76 (6H, s), 3.86 (3H, s), 6.36 (1H, t, J=2.0 Hz), 6.71 (2H, d, J=2.0 Hz), 6.76 (2H, d, J=8.8 Hz), 6.93 (1H, d, J=16.4 Hz), 7.15 (1H, d, J=16.4 Hz), 7.41 (2H, d, J=8.4 Hz), 7.99 (1H, s), 9.59 (1H, s); with DSC exhibiting a sharp endotherm at 117.30° C.;
e) Paraxanthine: Pterostilbene (1:1) characterized by ¹HNMR (400 MHz, dmso-d6): δ 3.17 (3H, s), 3.76 (6H, s), 3.85 (3H, s), 6.36 (1H, s), 6.71 (2H, s), 6.76 (2H, d, J=8.4 Hz), 6.94 (1H, d, J=16.8 Hz), 7.15 (1H, d, J=16.4 Hz), 7.41 (2H, d, J=8.0 Hz), 7.92 (1H, s), 9.59 (1H, s), 11.84 (1H, s), PXRD: 11.31 (12.8%), 15.88 (71.8%), 19.86 (18.7%), 21.28 (26.0%), 22.52 (33.6%), 24.9 (83.2%), 26.96 (100%), 29.00 (12.3%), 29.21 (19.3%), 30.41 (14.3%), 32.45 (9.9%), 38.8 (6.5%), 40.68 (5.2%), 44.84 (4.9%), 50.55 (3.6%), 55.48 (4.4%)±0.2° 2θ. DSC: A sharp endotherm observed at 204.17° C.;
f) Theophylline: Resveratrol (1:1) characterized by ¹HNMR (400 MHz, dmso-d6): δ 3.24 (3H, s), 3.44 (3H, s), 6.12 (1H, t, J=2 Hz), 6.39 (2H, d, J=2 Hz), 6.75 (2H, d, J=8.4 Hz), 6.81 (1H, d, J=16.4 Hz), 6.92 (1H, d, J=16.4 Hz), 7.38 (2H, d, J=8.4 Hz), 8.04, (1H, s) 9.21 (2H, s), 9.54 (1H, s), 13.57 (1H, s); with DSC exhibiting a sharp endotherm at 201.95° C.;
g) Theacrine: Pterostilbene cocrystal polymorphic Form I, characterized by an X-ray powder diffraction pattern having one or more peaks with relative intensity at about 12.78 (100%), 14.18 (90.2%), 14.69 (17.9%), 15.55 (19.0%), 17.45 (11.8%), 19.04 (9.1%), 22.28 (12.0%), 23.46 (14.4%), 27.90 (5.0%), 28.80 (5.6%), 29.50 (7.3%), 31.50 (7.6%), 33.04 (10.1%), 38.90 (14.2%), 41.02 (6.1%), 41.93 (4.7%), 53.16 (4.5%), 72.73 (8.4%)±0.2° 2θ;
h) Theacrine: Pterostilbene cocrystal polymorphic Form II, characterized by an X-ray powder diffraction pattern having one or more peaks with relative intensity at about 10.98 (4.8%),13.06 (33.6%), 14.45 (65.4%), 15.09 (38.5%), 15.85 (32.3%), 17.71 (6.5%), 19.18,(13.9%), 20.21 (28.7%), 21.44 (6.8%), 22.58 (11.1%), 23.73 (9.5%), 26.40, (59.7%), 27.21 (100%), 28.29 (10.0%), 29.12 (2.7%), 29.87 (3.6%), 31.76 (5.8%), 32.10 (6.2%), 33.33 (6.1%), 35.73 (1%), 37.02 (1.5%), 39.20 (9.0%), 40.66, (1.0%), 45.00 (1.4%), 46.38 (1.8%), 47.41 (1.2%), 48.05 (1.7%), 54.61 (2.8%), 55.46 (2.4%) and 72.69 (2.5%)±0.2° 2θ;
i) Theacrine: Pterostilbene cocrystal polymorphic Form III, characterized by an X-ray powder diffraction pattern having one or more peaks with relative intensity at about 12.90 (64.2%), 14.32 (100%), 15.64 (13.3%), 17.45 (8.2%), 19.17 (6.6%), 21.69 (10.5%), 22.38 (7.9%), 23.58 (10.0%), 25.07 (7.0%), 26.19 (6.1%), 26.98,(9.6%), 28.86 (1.9%), 29.59 (2.8%), 31.58 (2.7%), 33.12 (3.7%), 38.98 (7%), 41.10 (1.4%), 43.62 (1.1%), 47.80 (1.0%), and 72.70 (2.7%)±0.2° 2θ;
j) Theacrine: Pterostilbene cocrystal polymorphic Form IV, characterized by an X-ray powder diffraction pattern having one or more peaks with relative intensity at about 12.98 (100%), 14.38 (97.8%), 15.72 (18.6%), 17.66 (14.9%), 19.38 (10.3), 21.72 (7.5%), 22.47 (9.1%), 23.69 (12.8%), 25.14 (8.1%), 26.16 (11.6%), 27.00,(17.2%), 28.03 (2.5%), 29.69 (4.4%), 30.83 (1.1%), 31.66 (4.3%), 33.25 (7.2%), 36.88 (1.1%), 39.12 (11.6%), 41.21 (2.1%), 43.62 (1.3%), 44.06 (1.7%), 44.83, (1.3%), 45.99 (1.0%), 47.30 (1.1%), 47.95 (1.9%), 49.04 (1.0%), 53.36 (1.5%), 72.68 (3.3%) and 88.31 (1.0%)±0.2° 2θ;
k) Theacrine: Pterostilbene cocrystal polymorphic Form V, characterized by an X-ray powder diffraction pattern having one or more peaks with relative intensity at about 12.83 (17.0%), 14.33 (100%), 15.72 (18.8%), 19.15 (3.5%), 20.06 (3.5%), 22.10 (8.8%), 23.55 (6.5%), 27.17 (5.8%), 31.73 (4.8%), 33.71 (2.0%), 37.88, (1.4%), 39.19 6.2%), 41.07 (1.4%), 41.54 (2.2%), 43.86 (1.0%)±0.2° 2θ;
l) Theobromine: Pterostilbene cocrystal polymorph Form I characterized by an X-ray powder diffraction pattern having one or more peaks with relative intensity at about 11.75 (30.21), 13.38, (63.19), 15.3 (77.89), 17.31 (10.20%), 19.35 (95.4%), 23.46 (44.32%), 26.98 (100%), 29.35 (14.13%), 31.65 (6.50%), 34.30 (2.19%), 35.93 (6.20%), 39.15 (5.40%), 40.67 (3.61%), 42.08 (3.2%), 44.61 (2.87%), 48.89 (2.4%), 50.06 (2.9%), 52.53 (2.36%), 55.83 (2.22%), 72.73 (6.96%), 88.42 (2.35%);
m) Methylliberine: Pterostilbene cocrystal Form I, which is characterized by an X-ray powder diffraction pattern having one or more peaks at about 12.25 (87.7%), 13.33 (100%), 15.52 (24.8%), 16.44 (6.6%), 17.98 (6.0%), 19.99 (16.5%), 21.12 (8.6%), 21.99 (5.2%), 23.93 (4.5%), 25.44 (6.6%), 27.05 (11.1%), 28.76 (3.9%), 34.75 (3.8%), 37.81 (5.8%), 40.49 (3.0%), 42.78 (4.9%), 44.64 (3.9%), 72.73 (5.8%)±0.2° 2θ;
n) Methylliberine: Pterostilbene cocrystal Form II, which is characterized by an X-ray powder diffraction pattern having one or more peaks with relative intensity at about 12.43 (68.3%), 13.49 (100%), 15.65 (11.9%), 16.60 (11%), 18.12 (4.6%), 20.13 (15.3%), 21.30 (7.2%), 22.16 (6.2%), 23.34 (1.5%), 24.08 (4.4%), 25.69,(17%), 26.91 (16.8%), 27.31 (22.4%), 29.91 (1.3%), 30.82 (1.2%), 31.29 (1.6%), 34.51 (1.3%), 36.24 (1.4%), 37.94 (3.7%), 42.82 (1.0%), 44.67 (1.1%), 72.70 (2.5%)±0.2° 2θ;
o) Methylliberine: Pterostilbene cocrystal Form III, which is characterized by an X-ray powder diffraction pattern having one or more peaks with relative intensity at about 13.20 (100%), 15.35 (16.1%), 16.50 (11.8%), 17.90 (3.2%), 19.88 (13.3%),20.83 (3.9%), 22.06 (5.0%), 23.05 (1.3%), 23.88 (4.1%), 25.60 (14.9%), 36.03 (1.1%), 42.05 (1.2%), and 72.67 (7.7%)±0.2° 2θ;
p) Methylliberine: Pterostilbene cocrystal Form IV, which is characterized by an X-ray powder diffraction pattern having one or more peaks with relative intensity at about 12.00 (43.3%), 12.48 (91.5%), 13.63 (84.8%), 15.32 (11.0%), 16.77 (24.7%), 18.34 (10.8%), 19.02 (11.1%), 20.22 (20.4%), 21.44 (16.9%), 22.32, (14.8%), 23.34 (3.9%), 24.24 (10.5%), 25.84 (100%), 27.48 (81.2%), 33.31 (2.7%), 34.78 (5.1%), 35.01 (4.9%), 36.44 (3.5%), 38.10 (8.2%), 40.78 (1.5%), 43.12 (4.9%), 46.52 (2.7%), 47.75 (1.7%), 49.85 (1.4%)±0.2° 2θ;
q) Caffeine: Pterostilbene cocrystal Form I, characterized by an X-ray powder diffraction pattern having one or more peaks at 11.90 (28.6%), 15.03 (100%), 16.81 (51.2%), 19.06 (53.5%), 21.65 (44.4%), 25.81 (94.2%), 26.35 (91.04%), 28.16 (10.4%), 29.54 (6.7%), 31.32 (4.8%), 32.52 (10.3%), 35.74 (3.6%), 36.99 (3.8%), 39.36 (3.7%), 40.93 (7.3%), 43.66 (5.7%), 45.45 (3.6%), 49.83 (2.1%), 72.74 (8.5%) and 88.35 (3.3%)±0.2° 2θ;
r) Caffeine: Pterostilbene cocrystal polymorphic Form II, characterized by an X-ray powder diffraction pattern having one or more peaks with relative intensity at about 12.83 (2.19%), 17.04 (5.43%), 19.41 (5.24%), 22.25 (6.28%), 26.31 (100%), 28.67 (3.8%), 30.04 (1.6%), 32.96 (2.3%), 41.34 (1.1%), and 53.94 (1.7%)±0.2° 2θ;
s) Caffeine: Pterostilbene cocrystal polymorphic Form III, characterized by an X-ray powder diffraction pattern having one or more peaks with relative intensity at about 10.53 (18.3%), 12.18 (4.9%), 14.01 (23.04%), 15.56 (25.3%), 17.22 (21.0%), 19.35 (16.7%), 22.13 (13.7%), 24.02 (4.3%), 26.51 (100%), 28.57 (20.4%), 30.22 (4.0%), 31.82 (4.1%), 36.23 (1.1%), 43.3 (1.8%), 46.52 (1.1%), 53.52 (1.1%) and 72.72 (2.2%)±0.2° 2θ;
t) Caffeine: Pterostilbene cocrystal polymorphic Form IV, characterized by an X-ray powder diffraction pattern having one or more peaks with relative intensity at about 12.00 (43.3%), 12.48 (91.5%), 13.63 (84.8%), 15.32 (11.0%), 16.77 (24.7%), 18.34 (10.8%), 19.02 (11.1%), 20.22 (20.4%), 21.44 (16.9%), 22.32, (14.8%), 23.34 (3.9%), 24.24 (10.5%), 25.84 (100%), 27.48 (81.2%), 33.31 (2.7%), 34.78 (5.1%), 35.01 (4.9%), 36.44 (3.5%), 38.10 (8.2%), 40.78 (1.5%), 43.12 (4.9%), 46.52 (2.7%), 47.75 (1.7%), 49.85 (1.4%)±0.2° 2θ;
u) Paraxanthine: Pterostilbene cocrystal, characterized by an X-ray powder diffraction pattern having one or more peaks with relative intensity at about PXRD: 11.31 (12.8%), 15.88 (71.8%), 19.86 (18.7%), 21.28 (26.0%), 22.52 (33.6%), 24.9 (83.2%), 26.96 (100%), 29.00 (12.3%), 29.21 (19.3%), 30.41 (14.3%), 32.45 (9.9%), 38.8 (6.5%), 40.68 (5.2%), 44.84 (4.9%), 50.55 (3.6%), 55.48 (4.4%)±0.2° 2θ;
v) Theophylline: Resveratrol cocrystal, characterized by an X-ray powder diffraction pattern having one or more peaks with relative intensity at about PXRD: 10.30 (9.4%), 12.40 (30.7%), 13.81 (100%), 15.21 (9.4%), 16.27 (14.1%), 17.21 (4.5%), 19.22 (21.1%), 20.76 (13.0%), 22.30 (8.4%), 23.52 (8.4%), 24.84 (47.9%), 25.36 (33.6%), 26.64 (7.4%), 28.26 (8.6%), 30.68 (8.9%), 32.88 (3.3%), 34.96 (1.1%), 36.46 (1.5%), 37.61 (1.1%), 38.45 (2.0%), 39.84 (1.4%), 42.16 (2.3%), 45.91 (1.3%), 52.26 (1.3%), 72.73 (4.1%) and 88.35 (1.2%)±0.2° 2θ.

52. A process for preparation of the cocrystals or polymorphic forms as claimed in claim 50 comprising:
(a) dissolving the coformer Stilbenoid in a solvent at 35-55° C. to obtain clear solution followed by addition of Methylxanthine;
(b) stirring the above mixture at a temperature of 40-60° C. to obtain a clear solution.
(c) gradually cooling the above solution to ambient temperature and maintaining said temperature for a period of 10-20 h to allow precipitation of the cocrystal; and
(d) separating the co-crystal formed on standing followed by filtering, washing and drying.

53. The process as claimed in claim 52, wherein the solvent is selected from C1-C5 alcohols, water, acids selected from formic acid, acetic acid, propanoic acid, butyric acid; ethers; esters; ketones and the like alone or mixtures thereof.

54. A process for preparation of the cocrystals as claimed in claim 50, wherein said cocrystals may be prepared by mechanical grinding the mixture of Methylxanthine and coformer Stilbenoid to obtain the desired cocrystal.

55. The process as claimed in claim 54, wherein the Methylxanthine is selected from Theacrine, Theobromine, Methylliberine, Caffeine, Paraxanthine and Theophylline and the like alone or mixtures thereof; the Stilbenoid is selected from Pterostilbene or Resveratrol, preferably in 1:1 ratio.

56. A nutraceutical or a pharmaceutical composition comprising an effective amount of the cocrystals or polymorphic forms of Methylxanthine with coformer Stilbenoid as claimed in claim 50 and one or more acceptable excipients.

57. The composition as claimed in claim 56, wherein the excipients are selected from one or more of one or more of a binder, filler, lubricant, emulsifier, suspending agent, sweetener, preservative, buffer, wetting agent, disintegrant, diluents, binders, flavours, colours, suitable polymers effervescent agent, additive, and mixtures thereof.

58. The composition as claimed in claim 57, wherein the additive is selected from the group consisting of microcrystalline cellulose, lactose, sucrose, fructose, glucose, dextrose, dibasic calcium phosphate, calcium sulfate, cellulose, methylcellulose, cellulose derivatives, kaolin, mannitol, lactitol, maltitol, xylitol, sorbitol, sugar alcohols, dry starch, dextrin, maltodextrin, polysaccharides, and mixtures thereof.

59. The nutraceutical or the pharmaceutical composition as claimed in claim 56, wherein the composition is in the form of dosage form selected from the group consisting of a tablet, capsule, powder, suspension, and lozenge.

60. A pharmaceutical composition comprising an effective amount of the cocrystals or polymorphic forms of Methylxanthine with coformer Stilbenoid as claimed in claim 50 and one or more acceptable excipients for dermatological use.
